(19) United States
(12) Patent Application Publication
(10) Pub. No.: US 2026/0351354 A1
(43) Pub. Date: Nov. 5, 2026

(54) GENERATING A COMMON SUBSTRUCTURE DATA TREE TO REPRESENT MOLECULAR STRUCTURES TO SELECT TARGET MOLECULES FOR DRUG DISCOVERY

(71) Applicant: Recursion Pharmaceuticals, Inc., Salt Lake City, UT (US)

(72) Inventors: Oscar Charles WYATT, Wells (GB); Robert Thomas SMITH, Cambridge (GB)

(21) Appl. No.: 19/198,527

(22) Filed: May 5, 2025

(51) Int. Cl. G16C 20/50; G06N 3/092; G16C 20/70

(52) U.S. Cl. CPC G16C 20/50; G06N 3/092; G16C 20/70

(57) ABSTRACT

The present disclosure relates to systems, non-transitory computer-readable media, and methods that generates a common substructure data tree to further generate a set of target molecular structures. Indeed, in one or more implementations, the disclosed systems generate a common substructure data tree that includes a plurality of data tree branches and data leaves that corresponds to a plurality of molecular structures. Moreover, the disclosed systems generate the set of target molecular structures that align to a reward configuration by selecting molecular structures from the common substructure data tree. In particular, the disclosed systems use a multi-armed bandit model to select from the common substructure data tree based on a number of leaves previously selected from a data tree branch.

### BACKGROUND

Recent years have seen significant developments in hardware and software platforms for utilizing models to explore complex feature spaces. For example, conventional systems train models to sample complex structures such as molecular compounds from a feature space. Despite these recent advances, conventional systems suffer from a number of technical deficiencies, particularly with regard to accuracy, efficiency, and operational inflexibility in selecting molecular structures in complex feature spaces.

### SUMMARY

Embodiments of the present disclosure provide benefits and/or solve one or more of the foregoing or other problems in the art with systems, non-transitory computer-readable media, and methods for exploring a chemical space in a systemic fashion to select molecular structures in a diverse and exploitive manner. Specifically, the disclosed systems can generate a common substructure data tree (e.g., maximum common substructure tree) and use a multi-armed bandit model to explore a chemical space. For example, the disclosed systems generate a set of target molecular structures by selecting molecular structures from a common substructure data tree based on a measure of reward and/or a number of visits to a certain branch of the common substructure data tree (e.g., the disclosed systems learn from experience from what has already been selected). In particular, the disclosed systems seek to balance exploration with exploitation to identify molecular structures that align to a reward configuration (e.g., an objective).

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates an overview diagram of a common substructure exploration system generating a set of target molecular structures from a common substructure data tree in accordance with one or more embodiments.

FIG. 2A illustrates an example diagram of the common substructure exploration system selecting a data leaf from a data tree branch in accordance with one or more embodiments.

FIG. 2B illustrates an example diagram of the common substructure exploration system evolving and mutating a set of target molecular structures in accordance with one or more embodiments.

FIG. 3 illustrates an example diagram of the common substructure exploration system generating a measure of reward aligned with a reward configuration in accordance with one or more embodiments.

FIG. 4 illustrates an example diagram of the common substructure exploration system recording prior iterations of selecting a molecular structure from the common substructure data tree in accordance with one or more embodiments.

FIG. 5 illustrates an example diagram of the common substructure exploration system using the multi-armed bandit model to choose between reward and exploration in accordance with one or more embodiments.

FIG. 6 illustrates an example diagram of the common substructure exploration system learning from past iterations to determine a current selection of a molecular structure from the common substructure data tree in accordance with one or more embodiments.

FIG. 7 illustrates an example diagram of the common substructure exploration system generating a project reward distribution of selected molecular structures in accordance with one or more embodiments.

FIG. 8 illustrates an example diagram of the common substructure exploration system determining a directionality of an updated project reward distribution in accordance with one or more embodiments.

FIG. 9 illustrates an example environment of the common substructure exploration system in accordance with one or more embodiments.

FIG. 10 illustrates an example series of acts to generate a set of target molecular structures in accordance with one or more embodiments.

FIG. 11 illustrates a block diagram of a computing device for implementing one or more embodiments.

### DETAILED DESCRIPTION

Embodiments of the present disclosure provide benefits and/or solve one or more of the foregoing or other problems in the art with systems, non-transitory computer-readable media, and methods for a common substructure exploration system that identifies a set of target molecular structures for compound drug design/exploration purposes using a common substructure data tree. In one or more embodiments, the common substructure exploration system generates a common substructure data tree (e.g., a maximum common substructure tree) and then explores the chemical space as a multi-armed bandit problem utilizing the common substructure data tree. In particular, the common substructure exploration system 100 generates the common substructure data tree that represents a plurality of molecular structures and contains multiple data tree branches that further include multiple data tree leaves. For example, the common substructure data tree has data tree branches that indicate common molecular substructures of data tree leaves with the data tree branches.

As just mentioned, the common substructure exploration system 100 can explore the chemical space by treating the chemical space as a multi-armed bandit problem to balance exploration/exploitation, and to learn from previous experience. For example, the common substructure exploration system 100 prioritizes visiting data tree branches of the common substructure data tree that have not been previously visited and in some instances, further chooses to visit tree branches with a greater expected maximum reward (e.g., exploits reward). In some embodiments, the common substructure exploration system 100 selects a set of target molecular structures from the common substructure data tree and further uses one or more molecules from the set of target molecular structures for mutation, crossover, and scoring (e.g., scoring to filter down molecules from the target set for drug discovery purposes).

In one or more embodiments, the common substructure exploration system 100 can also model maximum expected reward/regret relative to a particular reward configuration (e.g., maximizing solubility, minimizing molecular weight, maximizing binding energy, etc.). In particular, the common substructure exploration system 100 models the expected reward/regret to more intelligently guide the exploration process, estimate directional changes across iterations of selecting molecular structures from the common substructure data tree, and select stopping conditions. For example, the common substructure exploration system 100 can utilize a Bayesian approach to generate a project reward distribution of rewards and determine a threshold project reward.

Further, by comparing project rewards for generated compounds relative to a project reward distribution, the common substructure exploration system 100 can update the project reward distribution. Moreover, the common substructure exploration system 100 can intelligently estimate the additional improvements to be gained by additional iterations and apply thresholds for determining stopping conditions. Furthermore, as the project distribution of rewards changes across iterations, the common substructure exploration system 100 can determine the direction that the distribution is trending and updated probabilities for producing improved molecules.

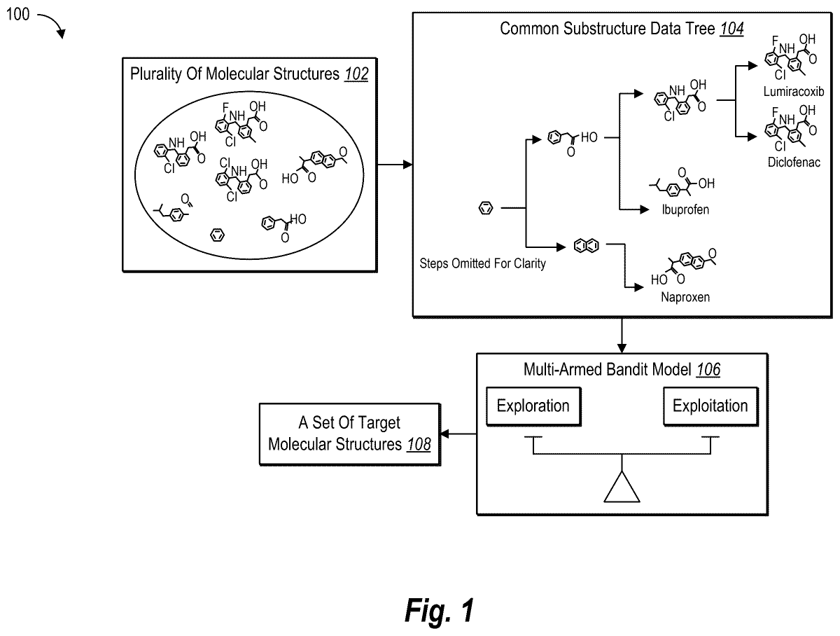

FIG. 1 illustrates the common substructure exploration system 100 generating a set of target molecular structures 108 from a common substructure data tree 104 that represents a plurality of molecular structures 102. For example, FIG. 1 shows the common substructure exploration system 100 accessing/receiving the plurality of molecular structures 102. In particular, the common substructure exploration system 100 accesses the plurality of molecular structures 102 from a database or library of molecules.

In one or more embodiments, the common substructure exploration system 100 accesses/receives the plurality of molecular structures 102 from a library of molecules. In particular, the library of molecules includes many different sets of molecular structures that are related to compound drug design/lead optimization. In some embodiments, the common substructure exploration system 100 receives a selection of a set of molecular structures from a user of a computing device (e.g., the user via the computing device selects the set of molecular structures from the library of molecules).

In one or more embodiments, the common substructure exploration system 100 receives an input molecule (e.g., a single molecule) from a user of a computing device (e.g., to initiate the process of molecular structure design for drug design purposes). In particular, the common substructure exploration system 100 processes the input molecule and uses it to select a set of molecular structures from the library of molecules (e.g., based on structural features and chemical features of the input molecule).

In some embodiments, in response to receiving the input molecule, the common substructure exploration system 100 utilizes a machine learning model to parse through a library of molecules (e.g., that includes molecular structures with a wide range of structural features and chemical features and/or chemical fingerprints of molecules) to identify chemical and structural features that are similar to the input molecule. In other words, the common substructure exploration system 100 can leverage an input molecule to diversify a starting population by swapping out certain groups in an input molecule with additional groups (e.g., the common substructure exploration system 100 can maintain the same core molecular substructure but with different R-groups).

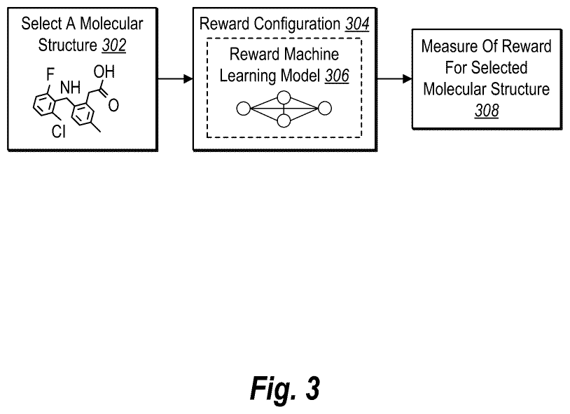

In some embodiments, the common substructure exploration system 100 utilizes a machine learning model to diversify the plurality of molecular structures 102. In particular, the common substructure exploration system 100 utilizes a machine learning model to diversify a starting population and the machine learning model is trained for a specific reward configuration. The reward configuration is discussed in more detail below in the description of FIG. 3.

In one or more embodiments, a molecular structure refers to an arrangement of molecules and/or atoms. Specifically, a molecular structure refers to a synthesized molecular structure that can include types and quantities of atoms (e.g., carbon, hydrogen, nitrogen, oxygen, etc.), a specific bonding arrangement (e.g., single bonds, double bonds, triple bonds), functional groups (e.g., —OH, —NH₂, —COOH, etc.), and a three-dimensional geometry (e.g., a spatial orientation of the molecular structure). Further, the molecular structure includes properties such as topology, folding, and higher-order interactions between structures (e.g., protein complexes, nucleic acid-protein complexes, lipids, etc.).

As mentioned above, molecular structures can include a common molecular substructure (e.g., a core). In other words, a first molecular structure can share a common core with a second molecular structure. In one or more embodiments, the common molecular substructure refers to a diverse structural molecule (e.g., structurally, the molecular building block can come as an aromatic ring, a heterocycle, an aliphatic chain, etc.) that serves as a foundational unit for constructing a molecular structure (e.g., a complex chemical compound). For instance, a molecular building block (e.g., the common molecular substructure) includes one or more reactive functional groups (e.g., amine, carboxylic acid, halogen) that can react with other molecular building blocks.

As shown in FIG. 1, the common substructure exploration system 100 generates the common substructure data tree 104 from the plurality of molecular structures 102. In one or more embodiments, the common substructure data tree 104 refers to a graph structure arranged according to shared or common core molecular substructure in downstream branches. In other words, the common substructure exploration system 100 generates the common substructure data tree so that downstream branches/leaves share a common substructure defined by any particular branch. Thus, a first branch represents a shared substructure for downstream leaves from the first branch whereas a second branch represents a shared substructure for downstream leaves from the second branch.

In one or more embodiments, the common substructure exploration system 100 generates representations for each molecular structure of the plurality of molecular structures 102. In particular, the common substructure exploration system 100 represents a molecular structure as a two-dimensional structural formula (e.g., atoms are represented by chemical symbols and bonds are represented as lines between the atoms), as a three-dimensional structural formula (e.g., a representation of the shape of molecules in space), as a graph (e.g., the atoms are nodes and chemical bonds are edges), or as a molecular fingerprint (e.g., numerical or vector representations of the molecular structures).

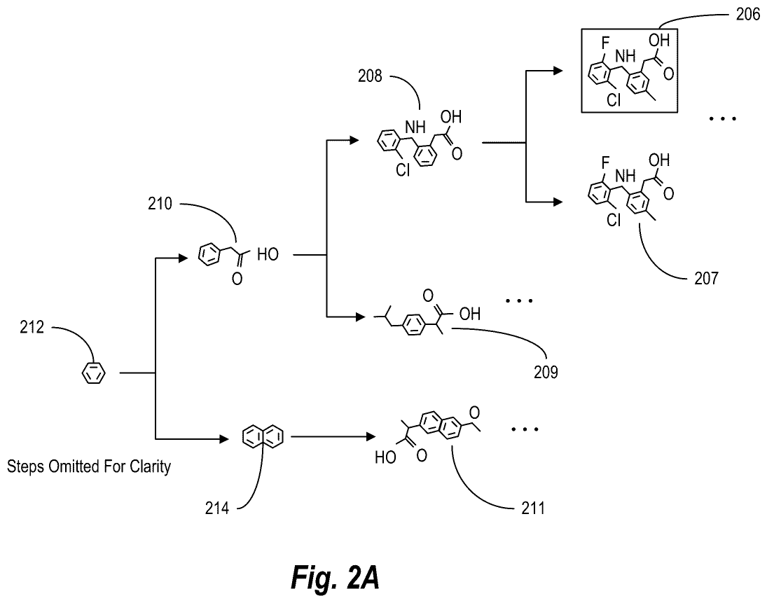

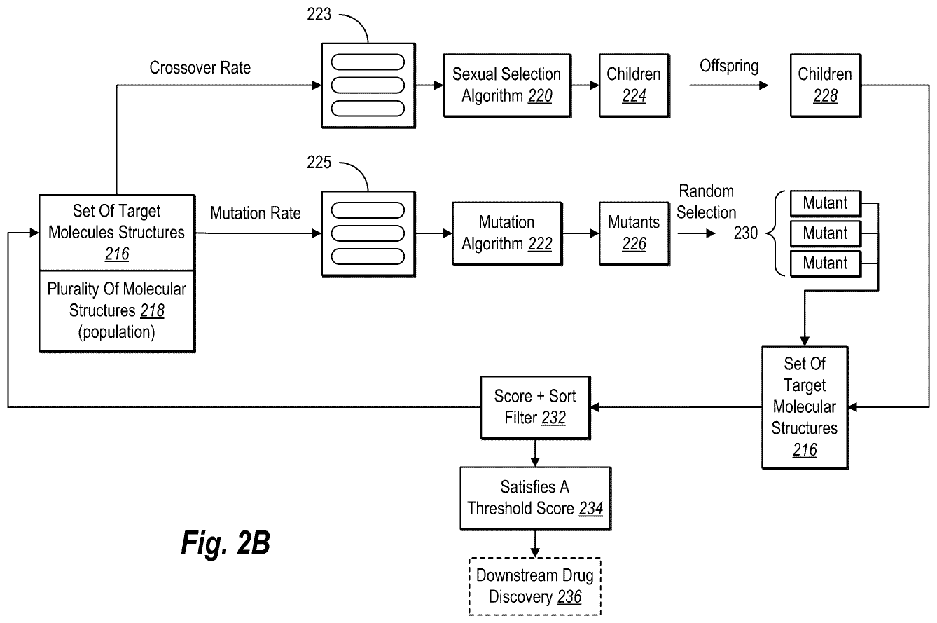

In some embodiments, the common substructure exploration system 100 utilizes generated representations of the plurality of molecular structures and attempts to find a maximum common substructure to identify the largest substructure shared by two or more chemical compounds (e.g., of the plurality of molecular structures). Further, the common substructure exploration system 100 compares representations (e.g., graphs) of the molecular structures to identify the largest fragment (e.g., common molecular substructure) that is common between the molecular structures. In other words, the common substructure exploration system 100 performs a similarity search amongst the plurality of molecular structures by using the maximum common substructure method to generate a common substructure data tree. In particular, the common substructure exploration system 100 matches the atoms (nodes) and bonds (edges) based on their connectivity and chemical properties (e.g., bond type and atom type). Additional details of generating the common substructure data tree 104 is given below in the description of FIG. 2A.

Moreover, FIG. 1 shows the common substructure exploration system 100 utilizing a multi-armed bandit model 106 to select molecular structure(s) from the common substructure data tree 104. In one or more embodiments, the common substructure exploration system 100 utilizes the multi-armed bandit model 106 to determine which molecular structure to select from the common substructure data tree 104. In particular, the common substructure exploration system 100 utilizes the multi-armed bandit model 106 to balance between exploitation and exploration.

In some embodiments, the common substructure exploration system 100 uses the multi-armed bandit model 106 to maximize total reward received by repeating selection actions (e.g., selecting molecular structures) from a set of options (e.g., the common substructure data tree). In particular, the “arms” of the multi-armed bandit model 106 are analogous the set of options available in the common substructure data tree 104, where each arm has an unknown probability of resulting in a reward.

Moreover, in some embodiments, each time the common substructure exploration system 100 uses the multi-armed bandit model 106 to select a molecular structure from the common substructure data tree 104, the common substructure exploration system 100 generates or receives a measure of reward and attempts to maximize a cumulative reward over a set of iterations (e.g., generations) of selecting from the common substructure data tree 104. As mentioned above, the common substructure exploration system 100 balances exploration with exploitation. In other words, the common substructure exploration system 100 tries different arms to learn reward distributions and with information regarding reward distributions, the common substructure exploration system 100 further chooses to exploit certain arms with the highest observed average reward to maximize the payoff of a cumulative reward. Additional details of the multi-armed bandit model 106 are also given below in the description of FIG. 2A.

Furthermore, FIG. 1 shows the common substructure exploration system 100 utilizing the multi-armed bandit model 106 to generate the set of target molecular structures. In one or more embodiments, the common substructure exploration system 100 generates the set of target molecular structures 108 that align to a reward configuration. In particular, the common substructure exploration system 100 uses the multi-armed bandit model 106 to balance between exploitation and exploration and selects the set of target molecular structures 108 from the common substructure data tree 104 (e.g., over multiple iterations). In some embodiments, the set of target molecular structures 108 are molecular structures that satisfy an established threshold of reward. Moreover, the common substructure exploration system 100 can use the set of target molecular structures 108 to initiate drug discovery processes.

As mentioned briefly above, existing systems suffer from inaccuracies, inefficiencies, and operational inflexibilities. Existing systems tend to explore narrow areas of a chemical space. In other words, existing systems are narrow in their scope of compound design and tend to be single minded in trying to maximize reward.

In one or more embodiments, many existing systems perform drug discovery in a complex and protracted manner that involves several technical steps. In particular, existing systems typically require identification of a target involved in a disease (e.g., to ensure that a designed drug will effectively target that disease). Further, existing systems then identify molecules that can interact with the identified target by performing high-throughput screening of massive chemical libraries (e.g., libraries containing thousands or millions of compounds). Moreover, via the high-throughput screening, existing systems then identify a set of compounds that interact with the target, and existing systems further optimize the set of compounds to obtain “lead compounds.”

Existing systems suffer from inaccuracies and can require tens of thousands of iterations to identify a single pharmaceutical compound viable for therapeutic treatment. Indeed, existing systems may inaccurately parse through a large volume of compounds to identify a set of compounds that may interact with a target but do not address the underlying issue of treating a disease. The sheer volume of data required for existing systems to parse through presents accuracy challenges in itself, as existing systems tend to drift towards finding solutions that are already known.

Additionally, existing systems further suffer from inaccuracies at the hit to lead stage (e.g., optimizing compounds to obtain lead compounds). In particular, existing systems run into issues with a high rate of attrition. In other words, the majority of compounds identified by existing systems (e.g., using high-throughput screening) fail due to issues with potency, selectivity, or toxicity. For example, related to the above issues of inaccuracies, existing systems further suffer from inaccuracies at the hit to lead stage due to the lack of chemical diversity of novel compounds that are actually identified in the high-throughput screening stage. In other words, the lack of chemical diversity directly results in compounds that are not ideal for drug design objectives.

Moreover, existing systems tend to output a high number of false positives and false negatives. In particular, existing systems tend to identify compounds that appear effective in targeting a substrate but are not actually effective. Further, existing systems often overlook compounds that could have been effective. As a result, existing systems waste resources on false positives and false negatives, and cause inefficiencies in the overall process. Accordingly, existing systems typically iterate through an excessive number of rounds of hit to discovery before identifying viable compounds that interact with a target substrate.

As alluded to above, existing systems also suffer from significant inefficiencies. In particular, due to the higher rate of attrition of non-diverse structures, existing systems waste additional computational resources, experimental resources, and time in having to perform and re-perform certain steps of the drug design process.

Moreover, existing systems suffer from operational inflexibilities due to being constrained to the inefficient and inaccurate drug design process described above. In one or more embodiments, existing systems are rigidly confined to sorting through a large volume of molecules and attempting to optimize an identified set of molecules (e.g., constrained to conventional drug design programs). In particular, existing systems tend to use rigid processes to find molecules that interact with a target and end up identifying molecules that are similar in function and structure (e.g., lack diversity). Thus, the operational constraints of existing drug design processes to identify viable compounds further exacerbate efficiency and accuracy concerns.

Furthermore, conventional systems also suffer from technical problems that arises in the realm of computer modeling. Indeed, many existing systems utilize computer algorithms to attempt to identify novel compounds; however, these computer systems suffer from similar inaccuracy issues and lack chemical diversity. Indeed, existing computer models suffer from overfitting issues as existing models tend towards the paths of least resistance.

Existing computer systems also suffer from computational inefficiencies. For example, some systems utilize iterative mutation processes to modify input drugs and iterate towards drugs with certain characteristics. However, these processes often require extensive time and computational resources as mutation processes fail to learn or intelligently explore the feature space. Moreover, these systems often struggle to identify efficient stopping criteria and fail to learn from prior iterations. Thus, existing computational processes often go through excessive iterations that require significant computing time and power.

In addition, existing computer systems often utilize computer algorithms that identify unrealistic molecules. In particular, existing computer systems leverage computer algorithms that generate non-synthesizable molecules. Moreover, existing computer systems utilize computer algorithms that fail to reflect realistic conditions and end up generating overly complex molecular structures that are not suitable for drug discovery processes. As such, existing computer systems are further faced with inefficiencies of sorting through and eliminating unrealistic/overly complex molecular structures.

In one or more embodiments, the common substructure exploration system 100 improves accuracy, efficiency, and operational flexibility relative to existing systems. For example, the common substructure exploration system 100 improves accuracy of compound design by enhancing the accuracy of exploring a chemical space.

In contrast to existing systems, which tend to be single minded in maximizing reward and are narrow in their scope of exploration, the common substructure exploration system 100 can expand the scope of exploration by balancing between diversity and exploitation. In particular, the common substructure exploration system 100 can generate the common substructure data tree 104 that represents the plurality of molecular structures 102. Moreover, the common substructure exploration system 100 can select molecules from the common substructure data tree 104 using the multi-armed bandit model 106 that balances considerations of reward versus exploration.

In doing so, the common substructure exploration system 100 can select the set of target molecular structures 108 that align to a reward configuration. Moreover, from the process of using the common substructure data tree 104 to select molecules, the common substructure exploration system 100 can identify additional diverse and viable molecular compounds for compound drug design pipelines. In other words, the selected molecular structures contain key properties that align with an objective (e.g., maximizing binding energy with a target substrate), are diverse, and are further used for designing accurate therapeutic treatments.

As mentioned above, existing systems use complex and protracted processes for drug discovery/design. In particular, existing systems perform multiple steps (e.g., high-throughput screening and hit-to-lead) where various inaccuracies manifest due to the lack of chemical diversity or novel compounds that are identified from those steps. In one or more embodiments, the common substructure exploration system 100 selects a molecular structure from a data tree branch of the common substructure data tree 104 based on a number of leaves previously selected and/or a measure of reward for that data tree branch.

In other words, the common substructure exploration system 100 can analyze both reward and whether a certain path has been explored when determining whether to select a molecular structure. Utilizing this approach, the common substructure exploration system 100 can converge upon a set of molecules that contain desirable properties (e.g., align with a reward objective) but also represent diverse and novel structures. Furthermore, in some embodiments, the common substructure exploration system 100 performs simulated mutation, crossover, and scoring on the set of target molecular structures 108 to further identify the most desirable molecules for drug discovery purposes.

As such, the common substructure exploration system 100 can address technical issues of existing systems that lack diversity. Because of this, the common substructure exploration system 100 can obtain molecular structures that are more accurate for drug design/discovery purposes. Thus, in one or more embodiments, the common substructure exploration system 100 suffers from a lower rate of attrition (relative to existing systems) and more quickly identifies molecular structures that are viable for a certain target (e.g., the common substructure exploration system 100 identifies a viable therapeutic treatment for treating a disease).

Related to the accuracy improvements, the common substructure exploration system 100 can also improve efficiency relative to existing systems. For example, the common substructure exploration system 100 can reduce the number of false positives and false negatives from generating the common substructure data tree 104 that represents the plurality of molecular structures 102 and selecting molecular structures based on both reward and diversity.

In other words, the common substructure exploration system 100 can identify a set of target molecular structures that has more diversity (e.g., relative to existing systems) and as such are more effective in interacting with a target. In particular, the common substructure exploration system 100 can identify diverse structures that improve the chances of selecting a molecule that fulfills a desired objective. In some embodiments, the common substructure exploration system 100 selects molecules from the set of target molecular structures 108 or selects molecules after performing mutation and crossover on the set of target molecular structures 108, which leads to a larger pool of diverse and novel structures.

Moreover, in some embodiments, the common substructure exploration system 100 can generate a measure of reward/regret to determine whether to continue additional iterations of selecting from the common substructure data tree 104. In other words, the common substructure exploration system 100 can intelligently determine how beneficial additional rounds of selecting molecular structures (e.g., for evolution/mutation) would be in increasing a measure of reward (or reducing a measure of regret). Accordingly, the common substructure exploration system 100 does not have to iterate through excessive iterations and does not have to excessively perform and re-perform certain drug design steps (e.g., relative to existing systems).

Additionally, the common substructure exploration system 100 can also improve operational flexibility relative to existing systems. For example, rather than being confined to rigid conventional drug design programs, the common substructure exploration system 100 can design an efficient and accurate framework for selecting molecular structures and learning from previous steps of selection. In particular, the common substructure exploration system 100 generates the common substructure data tree 104 and uses the multi-armed bandit model 106 to improve the operational diversity of arriving at a solution to obtain accurate molecular structures to target certain disease mechanisms. For instance, a user of a drug design pipeline can select one or more of the molecular structures from the identified set of target molecular structures 108 (e.g., or from one or more descendants or mutants of the set of target molecular structures 108) and use it for initiating drug development pipelines.

The common substructure exploration system 100 also addresses technical problems arising in the realm of computer modeling. Indeed, as just discussed, the common substructure exploration system 100 can utilize a common substructure data tree and multi-armed bandit algorithm to explore the chemical feature space and balance exploration and exploitation. Thus, the common substructure exploration system 100 can more accurately identify therapeutic compounds than conventional computing systems.

Furthermore, the common substructure exploration system 100 operates more efficiently by utilizing a common substructure data tree. Indeed, the common substructure exploration system 100 can more efficiently explore the feature space in an intelligent manner that learns from previous iterations to identify additional branches to explore based on a balance of reward and exploration. Furthermore, the common substructure exploration system 100 can monitor progress relative to expected reward modeling. Using this approach, the common substructure exploration system 100 can intelligently determine stopping criteria and identify exploration progress and directionality within a particular feature space.

Moreover, the common substructure exploration system 100 operates more efficiently by generating a project reward distribution. In particular, the common substructure exploration system 100 generates a project reward distribution of a set of target molecular structures to provide context for a maximum expected score for a given objective and an expected level of regret of running additional iterations of selecting molecular structures. In some embodiments, the common substructure exploration system 100 can provide a reasonable expectation of an additional level of reward to be obtained by running additional rounds of selecting molecules. Moreover, the common substructure exploration system 100 can assist analysts such that that they do not prematurely terminate the process of identifying target molecular structures, thus improving upon existing computer algorithms (e.g., for drug discovery processes).

FIG. 2A illustrates an example diagram of the common substructure exploration system 100 generating a common substructure data tree and selecting a single molecular structure in a first iteration in accordance with one or more embodiments. Moreover, FIG. 2B shows the common substructure exploration system 100 performing evolution/mutation on a selected molecular structure (e.g., or multiple selected molecular structures) for one or more iterations in accordance with one or more embodiments.

FIG. 2A shows the common substructure exploration system 100 generating a common substructure data tree (e.g., maximum common substructure data tree) from a plurality of molecular structures. In particular, FIG. 2A shows a core substructure of the common substructure data tree (e.g., a common substructure of the plurality of molecular structures) as a Benzene ring.

In one or more embodiments, the common substructure exploration system 100 generates the common substructure data tree shown in FIG. 2A by performing a plurality of acts. For example, the common substructure exploration system 100 accesses a plurality of molecular structures and generates a representation of the plurality of molecular structures. In some embodiments, the common substructure exploration system 100 generates a graph of the plurality of molecular structure, where each node represents an atom of a molecular structure, and each edge represents bonds between the atoms.

Moreover, the common substructure exploration system 100 uses a data tree algorithm or a graph-based algorithm to iterate through each of the molecular structures and identify the largest isomorphic subtree common to all input trees (e.g., input molecules of the plurality of molecular structures). In other words, the common substructure exploration system 100 finds a core substructure between two or more molecular structures where the attributes (e.g., atoms) and structural relationships (e.g., bonds and arrangement of bonds) match. In particular, the common substructure exploration system 100 finds the largest isomorphic subtree that exists in both molecules without breaking connectivity of the molecules (e.g., otherwise a single atom or bond in a molecule would inevitably be shared amongst two molecules and would be pointless for identifying viable molecular structures).

In one or more embodiments, the common substructure exploration system 100 aligns matching nodes (e.g., atoms) and edges (e.g., bonds), and if multiple matches exist between molecular structures, the common substructure exploration system 100 selects the larger substructure (e.g., the substructure with the greater number of nodes and edges relative to the other substructures). Furthermore, the common substructure exploration system 100 stores the common substructure data tree (e.g., as parent-child relationships) to retrieve from (e.g., for selecting one or more molecular structures).

In one or more embodiments, the common substructure exploration system 100 utilizes a ScaffoldTree algorithm to arrange a plurality of molecular structures into a common substructure data tree. For example, the common substructure exploration system 100 utilizes the ScaffoldTree algorithm to perform a hierarchical classification of chemical scaffolds by pruning terminal side chains. In particular, the common substructure exploration system 100 iteratively removes rings to form scaffolds at the higher levels of a hierarchy tree. In some embodiments, the common substructure exploration system 100 first removes peripheral rings one by one until only one ring remains. Further, the common substructure exploration system 100 removes the bonds and atoms that are part of the removed ring, excluding atoms and bonds which are part of any other ring. In some embodiments, if the removal of a ring would lead to a disconnected structure, the common substructure exploration system 100 does not remove the ring.

To illustrate, the common substructure exploration system 100 implements the ScaffoldTree algorithm as described in Schuffenhauer, A., Ertl, P., Roggo, S., Wetzel, S., Koch, M. A., and Waldman, H., 2007, The scaffold tree—visualization of the scaffold universe by hierarchical scaffold classification, Journal of chemical information and modeling, 47(1), pp. 47-58.

In some embodiments, a data tree branch refers to a portion of the common substructure data tree defined by a node corresponding to a molecular substructure. In particular, data tree branches of the common substructure data tree indicate common molecular substructures of data leaves within the data tree branches. In other words, the common substructure data tree can contain multiple data tree branches and data tree sub-branches, where each data tree branch/data tree sub-branch can contain one or more data leaves that share a common chemical feature corresponding to the branch/sub-branch. In some implementations, each branch corresponds to a node that defines the particular shared common molecular structure of all downstream sub-branches and leaves. Accordingly, a data tree branch/data tree sub-branch contains molecular structures that share a common molecular substructure.

In some embodiments, a data leaf is an individual node of the common substructure data tree that corresponds to a viable molecular structure (e.g., a molecular structure that is selectable by the common substructure exploration system 100). In particular, if the common substructure data tree contains a root node 212 (e.g., a core common molecular substructure), the common substructure data tree can further include multiple data tree branches coming off of the root node 212. Moreover, a data leaf in the common substructure data tree refers to a molecular structure that is viable and selectable by the common substructure exploration system 100. Thus, the common substructure exploration system 100 can iterate through nodes of a common substructure data tree until it reaches a data leaf (e.g., a viable molecular structure). A data leaf node can also form additional branches/sub-branches in the common substructure data tree.

In one or more embodiments, a viable molecular structure refers to a complete, stable, and/or functional chemical entity (e.g., that contains appropriate functional groups, stereochemistry, and interactions required for biological activity, bioavailability, and pharmacokinetics). The common substructure exploration system 100 can search for viable molecular structures that fit a target biomolecule and manifests appropriate pharmacological properties (e.g., suitable solubility, stability, and permeability). Moreover, in some embodiments, the common substructure exploration system 100 searches for viable molecular structures that contain functional groups that promote specific interactions with a biological target (e.g., a target aligned with a reward configuration).

As shown in FIG. 2A, the common substructure exploration system 100 generates the common substructure data tree from the plurality of molecular structures that can include the data tree branches. As shown (e.g., by the ellipses in FIG. 2A), the data tree branches of the common substructure data can contain multiple downstream nodes, however for clarity purposes a data tree of hundreds or thousands of molecular structures are not shown. In particular, FIG. 2A shows the root node 212 and a first data tree branch that contains molecular structure 210, molecular structure 208, molecular structure 206, and molecular structure 207 (e.g., and additional downstream molecular structures as indicated by the ellipses). Further, FIG. 2A shows a second data tree branch that contains the molecular structure 210 and molecular structure 209 (e.g., and additional downstream molecular structures as indicated by the ellipses). Moreover, FIG. 2A shows a third data tree branch that contains molecular structure 214 and molecular structure 211 (e.g., and additional downstream molecular structures as indicated by the ellipses).

Additionally, FIG. 2A shows a sub-branch of the first data tree branch that contains the molecular structure 208, the molecular structure 206 and the molecular structure 207. Further, FIG. 2A shows a sub-branch of the second data tree branch as the molecular structure 209 and a sub-branch of the third data tree branch as the molecular structure 211. Moreover, in some embodiments, the data leaves are shown as the molecular structure 206, the molecular structure 207, the molecular structure 209, and the molecular structure 211 (e.g., as the aforementioned molecular structures are viable molecular structures).

As mentioned above, the common substructure exploration system 100 utilizes a multi-armed bandit model to select from the common substructure data tree. Assuming that the common substructure exploration system 100 has no baseline information regarding reward of molecular structures in the common substructure tree (e.g., shown in FIG. 2A), the common substructure exploration system 100 uses the multi-armed bandit model to randomly select an arm (e.g., a data leaf part of a data tree branch) to initiate the process.

In one or more embodiments, the common substructure exploration system 100 uses an upper confidence bound for trees (UCT) in multi-armed bandit problems to select from the common substructure data tree. For example, an upper confidence bound for trees refers to a tree search problem (e.g., common substructure data tree). In particular, the common substructure exploration system 100 modifies the multi-armed bandit model to select from a tree of decisions rather than from a fixed set of arms. In some embodiments, each node of the common substructure data tree acts like a multi-armed bandit problem (discussed above), and the common substructure exploration system 100 decides which branch (e.g., action) to expand to.

In some embodiments, the common substructure exploration system 100 starts at the root node 212 and uses the multi-armed bandit model modified by UCT to move down (e.g., from the root node 212) the common substructure data tree and decide which molecular structure to select (e.g., the common substructure exploration system 100 selects a viable molecular structure). For example, from the root node 212, since the common substructure exploration system 100 has not selected from any tree branches, the common substructure exploration system 100 assigns similar UCT values to all paths. To illustrate, the common substructure exploration system 100 utilizes the multi-armed bandit model modified by UCT as described in Kocsis, L. and Szepesvari, C., 2006 September, Bandit based monte-carlo planning, In European conference on machine learning (pp. 282-293), Berlin, Heidelberg: Springer Berlin Heidelberg.

In one or more embodiments, the common substructure exploration system 100 starts at the root node 212 (e.g., the benzene) and then moves from the root node 212 to the molecular structure 210. Further, in some embodiments, from the molecular structure 210, the common substructure exploration system 100 moves to the molecular structure 208. Moreover, FIG. 2A shows the common substructure exploration system 100 selecting the molecular structure 206 to initiate a selection process for drug discovery/design purposes because the molecular structure has not yet been explored and is a viable molecular structure.

In one or more embodiments, for a first iteration (generation) of selecting from a common substructure data tree, visit counts will be zero (i.e., the common substructure exploration system 100 has not yet selected a leaf to analyze). Accordingly, the common substructure exploration system 100 utilizes the UTC modified multi-armed bandit model to recursively and randomly decide which child node to select until it reaches a molecular structure. After selecting a molecular structure, the common substructure exploration system 100 updates the visit count of that molecular structure to one. In some embodiments, the update to the visit count will ensure that the top-level node that contains the molecular structure 206 (e.g., the molecular structure that was selected in FIG. 2A) will have a visit count of one, which will be considered in selecting a molecular structure for a second iteration. For example, the common substructure exploration system 100 can select molecular structures from unvisited data tree branches and update the visit counts by using the UCT modified multi-armed bandit model.

Furthermore, in some embodiments, the common substructure exploration system 100 generates/simulates a measure of reward for the molecular structure 206 selected from the first data tree branch 200. Moreover, based on the measure of reward for the molecular structure 206, the common substructure exploration system 100 updates the reward for the upstream nodes (e.g., nodes corresponding to the molecular structure 208, the molecular structure 210, and the root node 212). In other words, the common substructure exploration system 100 continually updates a measure of reward for a given data tree branch based on selected molecular structures in that data tree branch and the reward determined for the selected molecular structures. As mentioned above, details of generating the measure of reward are given below in the description of FIG. 3.

As mentioned above, the common substructure exploration system 100 performs evolution and/or mutation on selections of molecular structures from the common substructure data tree. FIG. 2B illustrates the common substructure exploration system 100 performing evolution and mutation on one or more selected molecular structures in accordance with one or more embodiments. As alluded to above, the common substructure exploration system 100 selects from the common substructure data tree and performs evolution/mutation on the one or more molecular structures to further enhance diversity/novelty of identified structures.

In some embodiments, in response to selecting one or more selected molecular structures from the common substructure data tree, the common substructure exploration system 100 creates additional molecular structures from the one or more molecular structures. In particular, the common substructure exploration system 100 modifies selected molecular structures according to an evolution and/or mutation algorithm. In some embodiments, a user of a computing device can indicate whether to use a sexual selection algorithm 220 and/or a mutation algorithm 222 to modify one or more selected molecular structures. Moreover, in some embodiments, a user of a computing device can specify specific portions of selected molecular structures to modify via evolution/mutation.

FIG. 2B shows a set of target molecular structures 216 (e.g., molecules selected from the common substructure data tree using the UCT modified multi-armed bandit model). In particular, as shown in FIG. 2B, the common substructure exploration system 100 generates the set of target molecular structures 216 from a plurality of molecular structures 218 as described above in FIG. 2A (e.g., the common substructure data tree represents the plurality of molecular structures 218).

In some embodiments, the common substructure exploration system 100 utilizes the UCT modified multi-armed bandit model to select from the common substructure data tree shown above in FIG. 2A, defined as:

$$UCT = {\overline{X}}_{J} + 2 {C}_{p} \sqrt{\frac{2 ln n}{{n}_{j}}}$$

Where Xⱼ represents a reward, Cₚ represents an exploration parameter and nⱼ represents a visit count to a particular data tree branch. In particular, the above formulation of UCT modified multi-armed bandit model skews the common substructure exploration system 100 to explore less-visited nodes. For example, the log-term shown in the above formulation is tied to the number of visits, which causes the UCT value to be higher for less-visited nodes, thus the common substructure exploration system 100 prioritizes selecting from less-visited nodes.

As shown in FIG. 2B, the common substructure exploration system 100 selects a first subset of molecular structures 223 from the set of target molecular structures 216. Moreover, FIG. 2B illustrates the common substructure exploration system 100 applying a crossover rate 219 to the first subset of molecular structures 223 by using the sexual selection algorithm 220. In one or more embodiments, the crossover rate 219 refers to a genetic operator to combine genetic information of two parent molecular structures to produce offspring. In particular, the crossover rate 219 simulates a biological process of recombination. Further, in some embodiments, the crossover rate 219 determines a frequency of recombining genetic material from parent molecular structures (e.g., a high crossover rate results in more diverse offspring and a low crossover rate results in less diverse offspring).

Further, FIG. 2B illustrates the common substructure exploration system 100 applying a mutation rate 221 to a second subset of molecular structures 225 by using the mutation algorithm 222. In one or more embodiments, the mutation rate 221 refers to a genetic operator that represents random changes in genetic codes of a molecular structure. In particular, the mutation rate 221 simulates a biological process of genetic mutation. Further, in some embodiments, the mutation rate 221 controls a probability of frequency of mutations occurring (e.g., a high mutation rate results in more random changes to the molecular structure).

In one or more embodiments, the common substructure exploration system 100 utilizes the sexual selection algorithm 220 to select two molecular structures based on their fitness for a particular problem (e.g., a reward configuration). Moreover, the common substructure exploration system 100 utilizes the sexual selection algorithm 220 to choose a crossover point (e.g., a random point along the molecular structure) from a set of chemical transformations. In particular, the common substructure exploration system 100 utilizes the chosen crossover point to split the molecular structures and swap parts between the molecular structures to create offspring according to a selected chemical transformations. In some embodiments, the common substructure exploration system 100 uses a single point crossover, a two-point crossover, or a uniform crossover (e.g., each substructure of the molecular structure is randomly selected and swapped).

As shown in FIG. 2B, the common substructure exploration system 100 utilizes the sexual selection algorithm 220 to generate children 224. Accordingly, the common substructure exploration system 100 utilizes the sexual selection algorithm 220 to recombine parts of the “parent” molecular structures such that the children 224 contain new combinations of functional groups, bond structures, or stereochemistry.

As also shown in FIG. 2B, the common substructure exploration system 100 utilizes the mutation algorithm 222 to apply the mutation rate 221 to the second subset of molecular structures 225. For example, the common substructure exploration system 100 utilizes the mutation algorithm 22 to introduce random changes into the molecular structures. In particular, the common substructure exploration system 100 applies mutations to maintain diversity and prevent a premature convergence to a suboptimal solution.

In some embodiments, the common substructure exploration system 100 utilizes the mutation algorithm 222 to select a random part of the molecular structure (e.g., an atom, bond, or functional group) to modify. In particular, the common substructure exploration system 100 can use the mutation algorithm 222 to substitute an atom, add or delete atoms, modify bonds between atoms, change one or more functional groups into a different functional group, or add random parameters to the molecular structures (e.g., adding noise that represents variations in bond length or bond angles). As shown, the common substructure exploration system 100 utilizes the mutation algorithm 222 to generate mutants 226.

FIG. 2B shows that in some embodiments, the common substructure exploration system 100 can further generate (offspring) additional children from the children 224 (e.g., children 228) and further randomly select one or more of the mutants 226 and/or generate additional mutants. In particular, the common substructure exploration system 100 can obtain a set of molecular structures (e.g., children 228, mutants 230, and the set of target molecular structures 216) and use them to perform an act 232. As shown in FIG. 2B, the common substructure exploration system 100 further performs the act 232 of scoring, sorting, and filtering molecular structures from the children 228, the mutants 230, and the set of target molecular structures 216.

In one or more embodiments, the common substructure exploration system 100 utilizes the sexual selection algorithm 220 and the mutation algorithm 222 to generate products (e.g., descendants) that are viable molecular structures (e.g., synthesizable molecules). In particular, the common substructure exploration system 100 fine-tunes/optimizes the sexual selection algorithm 220 and the mutation algorithm 222 to conform with viable molecular structure principles, which were discussed above.

As mentioned, the common substructure exploration system 100 performs the act 232 of scoring, sorting, and filtering the children 228, the mutants 230, and the set of target molecular structures 216 to generate measures of reward for the children 228, the mutants 230, and the set of target molecular structures 216 (e.g., according to a reward configuration). Additional details of the scoring (e.g., generating the measures of reward) is described below in FIG. 3.

In one or more embodiments, the sorting of the molecular structures (e.g., from the children 228, the mutants 230, and the set of target molecular structures 216) occurs after scoring the molecular structures. In particular, the common substructure exploration system 100 sorts the molecular structures according to their measures of reward. Moreover, in some embodiments, the common substructure exploration system 100 further filters the sorted molecular structures. In other words, the common substructure exploration system 100 removes molecular structures below a certain score and retains molecular structures above a certain score. In particular, the common substructure exploration system 100 retains molecular structures above a certain score to continue to utilize for evolution/mutation in subsequent iterations.

As shown in FIG. 2B, the common substructure exploration system 100 compares the scored molecular structures with a threshold score 234, and if the common substructure exploration system 100 determines that one or more molecular structures satisfies the threshold score 234, it is further selected for an act 236. In particular, the common substructure exploration system 100 performs the act 236 of initiating downstream drug discovery for molecular structures that satisfy the threshold score 234, as the threshold score 234 indicates a high potential of reward for developing the molecular structure for a given reward configuration.

As mentioned above, the common substructure exploration system 100 updates the common substructure data tree with reward measures based on scoring the children 228, the mutants 230, and the set of target molecular structures 216. In one or more embodiments, the common substructure exploration system 100 identifies the measure of reward for a selected molecular structure and measures of reward for downstream molecules (e.g., evolved/mutated molecules) and uses that to assign reward to a data tree branch of the common substructure data tree.

As mentioned above, the common substructure exploration system 100 can use an average mean reward of a selected molecular structure and related molecular structures (e.g., related via evolution or mutation). As such, a measure of reward assigned to a data tree branch shows the potential reward of further selecting a molecular structure from a data tree branch. In some embodiments, using the average mean reward improves the selection diversity of the common substructure exploration system 100. In some embodiments, the common substructure exploration system 100 combines the measures of reward for each potential path. In some embodiments, the common substructure exploration system 100 takes the highest measure of reward relative to the other measures of reward for each potential path.

As mentioned, in one or more embodiments, the common substructure exploration system 100 selects multiple molecular structures (e.g., rather than just a single molecular structure shown in FIG. 2A as the molecular structure 206) from the common substructure data tree within a single iteration (e.g., generation). In particular, the common substructure exploration system 100 selects a large set (e.g., fifty molecular structures) from the common substructure data tree. Moreover, in some embodiments, the common substructure exploration system 100 performs mutation and/or evolution for the selected molecular structures from the common substructure data tree for each iteration.

In some embodiments, the common substructure exploration system 100 selects a large set of molecular structures from the common substructure data tree, however the common substructure exploration system 100 selects a single molecular structure at a time and evolves/mutates the single molecular structure before selecting another molecular structure from the common substructure data tree (within a single iteration). Moreover, the common substructure exploration system 100 generates a measure of reward for the single selected molecular structure, evolved molecular structures and/or mutated molecular structures to update the reward in the common substructure data tree for the single selected molecular structure. Further, the common substructure exploration system 100 continues to select additional molecular structures from the common substructure data tree based on the updated reward. In other words, within a single iteration (generation), the common substructure exploration system 100 continually updates the rewards/visit counts for selecting a set of molecular structures to evolve/mutate.

Although not shown in FIG. 2B, in some embodiments, the common substructure exploration system 100 scores/generates measures of reward for the plurality of molecular structures 218. As already mentioned above, the common substructure exploration system 100 prioritizes selecting from unvisited nodes in the common substructure data tree. In one or more embodiments, the common substructure exploration system 100 selects the highest scoring unexplored nodes (e.g., rather than randomly selecting unvisited nodes). In particular, because the common substructure exploration system 100 scores all of the molecular structures in the common substructure data tree (e.g., the plurality of molecular structures 218), the common substructure exploration system 100 can leverage that information (e.g., the measures of reward) to choose the highest scoring unvisited nodes (e.g., nodes that have not been selected to perform further evolution/mutation). Accordingly, the common substructure exploration system 100 nudges the multi-armed bandit model towards higher scoring but unexplored nodes.

In one or more embodiments, the common substructure exploration system 100 limits population size (e.g., the plurality of molecular structures 218) in the common substructure data tree. In some embodiments, the common substructure exploration system 100 limits the population size in later iteration rounds of selecting from the common substructure data tree. In particular, the common substructure exploration system 100 recreates a population with elite selection coupled with crossover and mutation selection. In some embodiments, including all of the plurality of molecular structures 216 from every generation results in a population of tens of thousands of molecules. As such, the common substructure exploration system 100 generates a common substructure data tree and can become bogged down in exploration (e.g., the common substructure exploration system 100 is unable to exploit its knowledge).

In some embodiments, the common substructure exploration system 100 reduces a size of the common substructure data tree so that more exploitation is possible. For example, the common substructure exploration system 100 prunes the population (e.g., the plurality of molecular structures 218) to a threshold size (e.g., an elite size) by sorting all molecules (e.g., the plurality of molecular structures 218) by reward and pruning the molecules that fail to satisfy a threshold (e.g., until a desired population size is reached).

In some embodiments, while limiting the population size, the common substructure exploration system 100 does not remove nodes from the common substructure data tree, but tags the nodes as pruned. In other words, the common substructure exploration system 100 maintains information about transformations/crossovers that result in high scoring/low scoring molecular structures, but it does not select from the pruned nodes. In doing so, the common substructure exploration system 100 can focus the chemical space to more “elite parts” e.g., higher scoring areas of the chemical space while still maintaining high levels of diversity.

Accordingly, the common substructure exploration system 100 can perform the processes shown in FIG. 2B to generate scores for a set of target molecular structures, identify “elite” molecular structures that satisfy the threshold score 234, and then prune the common substructure data tree to a more manageable population size for subsequent iterations. Moreover, in some embodiments, the common substructure exploration system 100 discounts a measure of reward for a pruned molecular structure when calculating a mean average reward for a data tree branch.

In one or more embodiments, the common substructure exploration system 100 selects a molecular structure in a first iteration and calculates a measure of reward (e.g., score) for sibling molecular structures of the selected molecular structure. In other words, the common substructure exploration system 100 generates a measure of reward for a selected molecular structure during an iteration (generation) and after an iteration has been completed. As mentioned above, after selecting the molecular structure in the first iteration, the common substructure exploration system 100 updates the visit counts during the selection, such that the common substructure exploration system 100 properly continues to explore the common substructure data tree.

As discussed above in FIG. 2A, the common substructure exploration system 100 selects the molecular structure 206 from the first data tree branch 200 and the molecular structure 206 further includes a sibling molecular structure (e.g., also part of the first data tree branch 200 and they share a parent in the tree). Based on a measure of reward generated for the molecular structure 206, the common substructure exploration system 100 further assigns sibling molecular structures a reward of the molecular structure 206. To illustrate, if the selected molecular structure has a reward of 0.7, then the common substructure exploration system 100 also assigns the sibling molecular structure a reward of 0.7, rather than a reward of 0 during the current iteration (e.g., to prevent artificially decreasing the likelihood of that entire branch being selected).

The pseudo code below illustrates the common substructure exploration system 100 assigning reward to sibling nodes:

| | |
|---|---|
| 1 | def reward(self) → float: |
| 2 | if self.has_scored_products( ): |
| 3 | Return self.score( ) |
| 4 | |
| 5 | return max([sibling.score( ) for sibling in self. siblings]) |
| 6 | |
| 7 | |
| 8 | def score(self) → float: |
| 9 | if self.has)scored_products( ): |
| 10 | return max([product.score( ) for product in self.scored_products]) |
| 11 | # if a node's products haven't been scored, but the molecule itself has |
| 12 | # return that score as it gives)some)indication of likely reward |
| 13 | # (i.e., its better than a score of 0) |
| 14 | if self.is_molecule( ) and self.has_been_scored( ): |
| 15 | return self.molecule_score( ) |
| 16 | return 0.0 |

In some embodiments, however, the common substructure exploration system 100 does not score all the molecular structures of the plurality of molecular structures 218. For example, FIG. 2B shows the common substructure exploration system 100 scoring from the children 228, the mutants 230, and the set of target molecular structures 216, instead of the plurality of molecular structures 218.

As mentioned, in embodiments the common substructure exploration system 100 only scores from the children 228, the mutants 230, and the set of target molecular structures 216. In particular, the common substructure exploration system 100 scores a subset (e.g., one or more) from the children 228, the mutants 230, and the set of target molecular structures 216. In some embodiments, the common substructure exploration system 100 decreases a visit count if none of a selected molecular structure's products (e.g., evolved or mutated structures) are scored (e.g., to increase the probability of selecting from that data tree branch to enhance exploration).

In one or more embodiments, the common substructure exploration system 100 does not select from unexplored nodes with the highest score. In particular, the common substructure exploration system 100 chooses a random unexplored node weighted by a score of each node. In other words, the common substructure exploration system 100 biases selection towards high scoring nodes, while still including an element of chance in order to boost diversity. In some embodiments, using weight probabilities towards higher scoring unexplored nodes further improves selection performance of the common substructure exploration system 100 for diversity and reward.

FIG. 3 illustrates an example diagram of the common substructure exploration system 100 using a reward configuration to generate a measure of reward for a selected molecular structure in accordance with one or more embodiments. For example, FIG. 3 shows that the common substructure exploration system 100 selects a molecular structure 302 (e.g., the molecular structure selected in FIG. 2A) and generates a measure of reward 308 for the molecular structure 302. In particular, the common substructure exploration system 100 generates the measure of reward 308 based on a reward configuration 304.

In one or more embodiments, the reward configuration 304 refers to an objective for a decision-making model (e.g., the common substructure exploration system 100 using the UCT modified multi-armed bandit model) to select a molecular structure from the common substructure data tree. In other words, the reward configuration 304 acts as a feedback mechanism to indicate whether the action (e.g., the selection of a molecular structure) was effective or ineffective relative to achieving its objective. In particular, the reward configuration 304 guides a decision-making process by quantifying rewards or penalties for different outcomes (e.g., selections of molecular structures).

To illustrate, the common substructure exploration system 100 uses reward configurations such as solubility, binding energy, molecular weight, a structure-phenomics relationship (“sphere”) system, digital phenomaps (e.g., improving phenomic similarity relative to a target gene or compound), and a molecular graph prediction system. In other words, the common substructure exploration system 100 selects a reward configuration to guide the selection of molecules for a specific purpose of designing a drug compound.

As mentioned, the common substructure exploration system 100 utilizes predicted phenomic similarity as a reward configuration. For example, the common substructure exploration system 100 can utilize a structure-phenomics relationship model as a reward configuration. In one or more embodiments, the common substructure exploration system 100 incorporates with the structure-phenomics relationship (“sphere”) system, which is described in patent application Ser. No. 18/753,906 DETERMINING PHENOMIC RELATIONSHIPS BETWEEN COMPOUNDS AND CELL PERTURBATIONS UTILIZING MACHINE LEARNING MODELS, which was filed on Jun. 25, 2024, and is fully incorporated by reference herein. In particular, a sphere system utilizes machine learning models to predict phenomic relationships between chemical compounds and cell perturbations (e.g., gene knockout sequences or other chemical compounds). For example, the sphere system can determine a structural feature representation for the chemical compound and generate a phenomic similarity prediction (e.g., a classification prediction for similarity in cell phenotype resulting from application of the chemical compound relative to one or more perturbations). The common substructure exploration system 100 can maximize or minimize phenomic similarity relative to one or more target genes or target compounds as a reward configuration.

Moreover, in some embodiments, the sphere system utilizes the phenomic similarity predictions in a drug discovery pipeline or other task. For example, the sphere system can utilize phenomic similarity predictions to select compounds for subsequent testing, to confirm structure-phenomic relationships with genes for mechanism of action analysis, to find chemical compounds with promising pheno-similarity characteristics, and/or to supplement a phenomap and actively update the machine learning model for enhanced phenomic similarity predictions. In other words, the sphere system acts a reward configuration (objective) that guides the common substructure exploration system 100 in selecting from a common substructure data tree. Thus, the common substructure exploration system 100 can generate a common substructure data tree and use the sphere system to aid in selecting compounds to confirm measures of reward for structure-phenomic relationships.

In one or more embodiments, the common substructure exploration system 100 incorporates with a perturbation mapping system to determine measures of reward for molecular structures selected from a common substructure data tree. For example, the common substructure exploration system 100 incorporates with the perturbation mapping system which is described in patent application Ser. No. 18/526,707 UTILIZING MACHINE LEARNING MODELS TO SYNTHESIZE PERTURBATION DATA TO GENERATE PERTURBATION HEATMAP GRAPHICAL USER INTERFACES, which was filed on Dec. 1, 2023, and is fully incorporated by reference herein.

In some embodiments, the perturbation mapping system synthesizes perturbation data by embedding perturbation images via a machine learning model and filtering, aligning, and aggregating the embeddings to generate a genome-wide perturbation database for real-time generation of perturbation heatmap interfaces. In particular, the perturbation mapping system can synthesize biological perturbation data (e.g., phenomic digital images portraying cells resulting from various perturbations), by embedding the data into a low dimensional feature space via a machine learning model.

Furthermore, in one or more implementations, the perturbation mapping system applies filtering, alignment, and aggregation models to generate accurate perturbation-level representations for compilation into a perturbation database. Indeed, utilizing this approach, the perturbation mapping system can accurately relate machine learning embeddings from any number of perturbation experiments for flexible comparison and analysis. Further, the perturbation mapping system can identify perturbation relationships by accessing the database, in response to a query of one or more perturbations, and determine a similarity measure between machine learning embeddings of the queried perturbations and the perturbations of the database. Additionally, the perturbation mapping system can generate, for display on a client device, an interactive heatmap of the identified perturbation relationships along with additional user interface elements for dynamic and efficient analysis

In some embodiments, the common substructure exploration system 100 can reference generated phenomaps from the perturbation mapping system for a reward configuration. In particular, the common substructure exploration system 100 can generate a measure of reward for molecular structures based on an indicated phenomap that indicates a level of similarity of a selected molecular structure with a target. In other words, the common substructure exploration system 100 draws from multiple data sources when generating a measure of reward for a particular molecular structure.

In one or more embodiments, the common substructure exploration system 100 incorporates with a molecular graph prediction system to determine measures of reward for selected molecular structures. In particular, the molecular graph prediction system is described in patent application Ser. No. 18/750,813 TRAINING AND UTILIZING COMPOUND GRAPH NEURAL NETWORKS TO GENERATE BIOLOGICAL ACTIVITY PREDICTIONS FROM INPUT CHEMICAL COMPOUNDS, which was filed on Jun. 21, 2024 and is fully incorporated by reference herein. In some embodiments, the molecular graph prediction system trains and utilizes a compound graph neural network architecture to generate biological activity predictions from input compounds. For example, the molecular graph prediction system can utilize a compound graph neural network to analyze an input compound and generate a variety of predictions for novel tasks, such as chemical activity predictions, compound program predictions, phenomic embedding predictions, and/or transcriptomic predictions. Thus, the common substructure exploration system 100 generates a common substructure data tree, selects one or more molecular structures, and uses the molecular graph prediction system to generate a measure of reward for the selected one or more molecular structures based on analyzing them with the compound graph neural network.

Moreover, the molecular graph prediction system can also extract fingerprints from pre-trained prediction heads to finetune and implement a compound graph neural network for generating additional or alternative predictions. For example, the molecular graph prediction system can initially train a compound graph neural network to generate a variety of quantum physics, chemistry, or biology tasks utilizing a first set of pre-trained prediction heads. The molecular graph prediction system can then finetune the compound graph neural network by extracting fingerprints from these pre-trained prediction heads (and/or extracting fingerprints from other pre-trained models) and utilize additional, efficient neural network layers to generate predictions for additional tasks. In this manner, the molecular graph prediction system can train and utilize compound graph neural networks to flexibly transform and utilize input compounds to generate accurate biological activity predictions. Similarly, the common substructure exploration system 100 can use the fine-tuned compound graph neural network to score molecular structures selected from the common substructure data tree (e.g., if the molecular graph prediction system is the selected reward configuration).

In one or more embodiments, based on drug design requirements, the common substructure exploration system 100 selects a reward configuration from a plurality of reward configurations. In particular, the plurality of reward configurations includes a plurality of objectives to improve in selecting target molecular structures from a common substructure data tree. For example, the common substructure exploration system 100 receives the reward configuration 304 from a user of a computing device that initially provides/submits a starting population of molecular structures (e.g., the plurality of molecular structures used to generate a common substructure data tree). In particular, a user of a computing device can select from a list of objectives that are tailored for a drug design process.

In one or more embodiments, based on a drug design purpose (e.g., designing a drug that targets type II diabetes, asthma, lymphocytes, etc.), a user of a computing device typically has a target biological substrate, pathway, or mechanism that they are attempting to effectively interact with. In other words, a user of a computing device provides a target biological substrate, pathway, and/or mechanism and further indicates an objective such as target protein binding, drug likeness, toxicity, solubility, pharmacokinetics, etc.

In some embodiments, in response to selecting the molecular structure 302, the common substructure exploration system 100 generates the measure of reward 308 by comparing the molecular structure to the reward configuration 304. In particular, for binding energy as the reward configuration, the common substructure exploration system 100 computes the reward measure by utilizing molecular docking simulations. In other words, the common substructure exploration system 100 utilizes simulation models to determine a binding energy of the molecular structure 302 interacting with a target.

In some embodiments, the common substructure exploration system 100 generates the measure of reward 308 for multiple objectives. In particular, the common substructure exploration system 100 receives the reward configuration 304 for binding energy and solubility. As such, the common substructure exploration system 100 utilizes multi-objective optimization models to combine different factors with weights and calculate a reward measure using a multi-factor reward function.

As shown in FIG. 3, in some embodiments, the common substructure exploration system 100 utilizes a reward machine learning model 306. In one or more embodiments a machine learning model includes a computer algorithm or a collection of computer algorithms that can be trained and/or tuned based on inputs to approximate unknown functions. For example, a machine learning model can include a computer algorithm with branches, weights, or parameters that changed based on training data to improve for a particular task. Thus, a machine learning model can utilize one or more learning techniques to improve in accuracy and/or effectiveness. Example machine learning models include various types of decision trees, support vector machines, Bayesian networks, random forest models, or neural networks (e.g., deep neural networks).

Similarly, a neural network includes a machine learning model of interconnected artificial neurons (e.g., organized in layers) that communicate and learn to approximate complex functions and generate outputs based on a plurality of inputs provided to the model. In some instances, a neural network includes an algorithm (or set of algorithms) that implements deep learning techniques that utilize a set of algorithms to model high-level abstractions in data. To illustrate, in some embodiments, a neural network includes a convolutional neural network, a recurrent neural network (e.g., a long short-term memory neural network), a transformer neural network, a generative adversarial neural network, a graph neural network, a diffusion neural network, or a multi-layer perceptron. In some embodiments, a neural network includes a combination of neural networks or neural network components.

In one or more embodiments, the common substructure exploration system 100 selects the molecular structure 302 from the common substructure data tree and uses the reward machine learning model 306 to generate the measure of reward 308 for the selected molecular structure. In particular, the common substructure exploration system 100 uses the reward machine learning model 306 to process the selected molecular structure and generate an output prediction for the selected molecule based on the reward configuration 304. In other words, the common substructure exploration system 100 uses the reward machine learning model 306 attuned for a specific objective (e.g., binding energy, solubility, etc.) and determines the reward for the selected structure.

In one or more embodiments, the common substructure exploration system 100 trains the reward machine learning model 306. In particular, the common substructure exploration system 100 uses training data that contains ground truth measures of reward. During training, the common substructure exploration system 100 uses the machine learning model to generate output predictions for certain molecules and compares the output predictions to ground truth measures of reward (e.g., determined/measured in a laboratory setting). Based on the comparison, the common substructure exploration system 100 modifies parameters of the machine learning model. In particular, the common substructure exploration system 100 optimizes multiple different machine learning models for different reward configurations.

In one or more embodiments, the common substructure exploration system 100 utilizes deep learning models to predict molecular properties of the molecular structure 302. In particular, the common substructure exploration system 100 utilizes deep learning models that have been optimized on data showing molecular structures and their corresponding outputs (e.g., outputs such as binding energy to similar targets or solubility in certain solutions). In doing so, the common substructure exploration system 100 utilizes deep learning models to generate predictions for the molecular structure 302. Moreover, based on the predictions of molecular properties, the common substructure exploration system 100 can further predict the measure of reward 308.

As mentioned above, the common substructure exploration system 100 generates the measure of reward 308 for the molecular structure 302. In one or more embodiments, the measure of reward 308 refers to a value that quantifies how well a model performs for a specific task or objective. For example, an agent model (e.g., the UCT modified multi-armed bandit model) makes decisions and receives feedback in the form of rewards, where the rewards indicate how desirable or undesirable an outcome of an action or a sequence of actions was. In some instances, the agent has an objective to maximize the reward.

As described above, for selecting molecular structures from the common substructure data tree, there can be a variety of objectives for a reward (e.g., prediction of a binding affinity to a specific protein, molecular properties such as stability and reactivity, predicting a binding affinity to a target transcription factor). In some instances, the common substructure exploration system 100 seeks to balance multiple objectives.

Although not shown in FIG. 3, in some embodiments, the common substructure exploration system 100 selects the molecular structure 302 and then performs evolution/mutation of the molecular structure 302. Further, the common substructure exploration system 100 generates a plurality of molecular structures (e.g., mutated/evolved) from the molecular structure 302 and then utilizes the reward configuration 304 to generate multiple measures of reward.

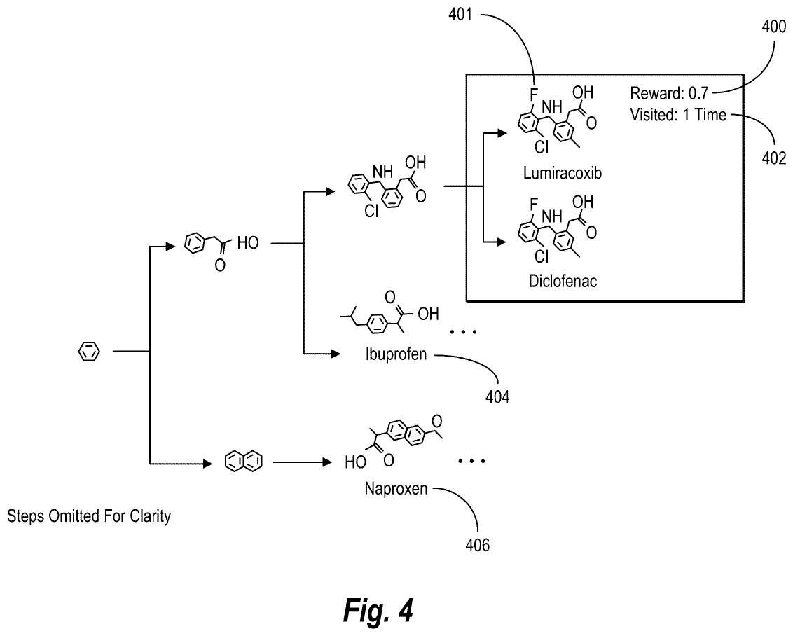

FIG. 4 illustrates an example diagram of the common substructure exploration system 100 using a reward and a number of visits to a data branch of the data tree to determine which additional molecular structure to select in accordance with one or more embodiments. For example, FIG. 4 shows an additional iteration (generation) of selecting from a common substructure data tree after a viable molecular structure from a data tree branch was selected in a previous round (e.g., as shown in FIG. 2A).

As shown in FIG. 4, the common substructure exploration system 100 selected a molecular structure from a data tree branch and generates a measure of reward 400 for the selected molecular structure. In particular, FIG. 4 shows the measure of reward 400 as 0.7. Moreover, FIG. 4 shows a number of visits 402 as one time. In one or more embodiments, the number of visits 402 indicates a number of times the common substructure exploration system 100 has selected from a data tree branch. In particular, the common substructure exploration system 100 considers the number of visits 402 to determine whether to explore other data tree branches of the common substructure data tree.

As mentioned above, in some embodiments, the common substructure exploration system 100 utilizes the UTC modified multi-armed bandit model to make decisions of selecting molecular structures from the common substructure data tree. In particular, the common substructure exploration system 100 utilizes the UTC modified multi-armed bandit model to balance exploration and exploitation. As mentioned, in some embodiments, the common substructure exploration system 100 references a number of visits to different data tree branches to prioritize exploring different molecular structures, and as such also prioritizes increasing the diversity/novelty of selected molecules.

As mentioned above, the common substructure exploration system 100 utilizes the UTC modified multi-armed bandit model to select a molecular structure 401 from a data tree branch (e.g., in a first iteration) and then uses a sexual selection algorithm and/or a mutation algorithm to generate children and mutants of the molecular structure 401. Furthermore, in some embodiments, the common substructure exploration system 100 then generates measures of reward for the molecular structure 401, the children, and/or the mutants. From the measures of reward, the common substructure exploration system 100 can further update the data tree branch to reflect an average mean reward or a max reward of the molecular structure 401, the children, and/or the mutants. In some embodiments for a second iteration, the common substructure exploration system 100 selects a data leaf 404 or a data leaf 406 to continue exploring the common substructure data tree (e.g., at this point, the common substructure exploration system 100 prioritizes diversity over exploitation).

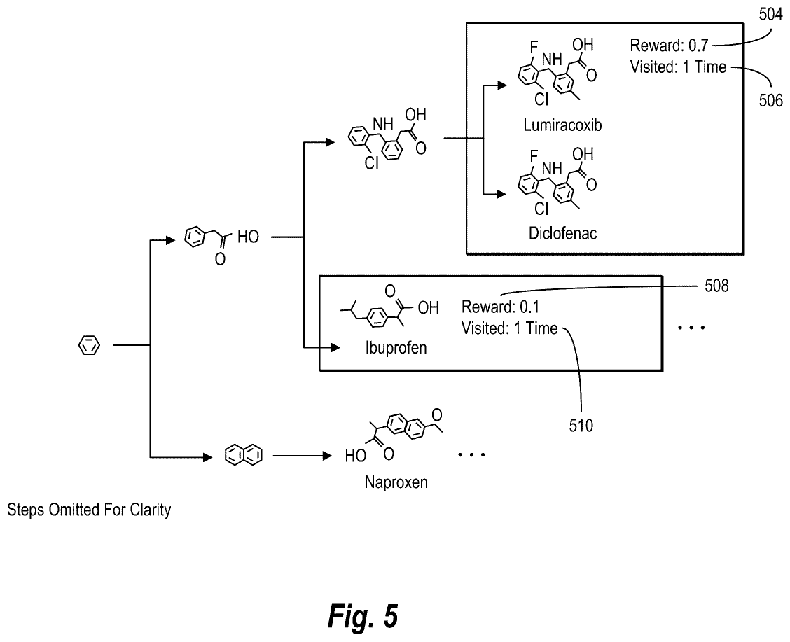

FIG. 5 illustrates an example diagram of the common substructure exploration system 100 utilizing the multi-armed bandit model to guide a decision-making process of selecting a viable chemical structure to add to a set of target molecular structures. In particular, FIG. 4 shows the common substructure exploration system 100 considering measures of reward and the number of visits for the data tree branches in a common substructure and determining which branch to exploit in accordance with one or more embodiments.

As mentioned above, the common substructure exploration system 100 utilizes the UTC modified multi-armed bandit model to balance exploration and exploitation. FIG. 5 illustrates the common substructure exploration system 100 considering measures of reward and a number of previous visits to determine which viable molecular structure to select. In particular, FIG. 5 shows a first measure of reward 504 (e.g., 0.7) and a first number of visits 506 (e.g., one visit) for a data tree branch (e.g., a data tree branch/a data tree sub-branch that includes Lumiracoxib and Diclofenac). Further, FIG. 5 shows a second measure of reward 508 (e.g., 0.1) and a second number of visits 510 (e.g., 1 time) for another data tree branch (e.g., that contains Ibuprofen).

In one or more embodiments, the common substructure exploration system 100 in a previous iteration (e.g., the iteration shown in FIG. 4) selected a molecular structure (e.g., Ibuprofen). As such, in some embodiments, the common substructure exploration system 100 determines which data tree branch to further exploit by holistically considering the generated measures of reward and the number of visits. In some embodiments, the common substructure exploration system 100 selects a data tree branch with a fewer number of visits to further exploit. For example, the common substructure exploration system 100 utilizes the UCT modified multi-armed bandit problem and determines to select from a data tree branch with the fewer number of visits (e.g., based on a principle increasing diversity of selected molecular structures).

As shown in FIG. 5, the first measure of reward 504 is greater than the second measure of reward 508 and the number of visits for both are the same (i.e., one time each). Accordingly, the common substructure exploration system 100 utilizes the UCT modified multi-armed bandit model to consider the measures of reward balanced by the number of visits and exploits a data tree branch with a higher potential for reward.

In one or more embodiments, FIG. 5 illustrates the common substructure exploration system 100 utilizes the UTC modified multi-armed bandit model to explore the first data tree branch 500 and then the second data tree branch 502, and rather than continuing exploration, the common substructure exploration system 100 seeks to prioritize exploitation.

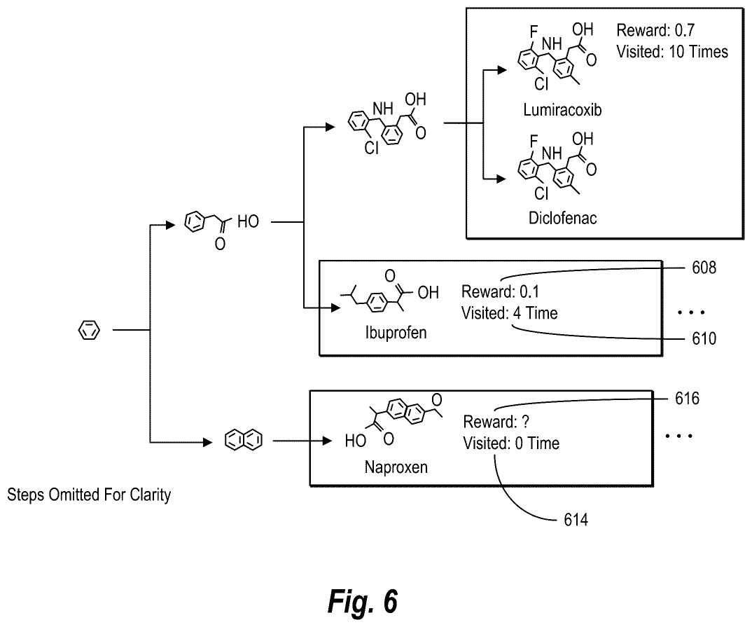

FIG. 6 illustrates an example diagram of the common substructure exploration system 100 comparing multiple data branches with each other to determine which branch to exploit in accordance with one or more embodiments. For example, FIG. 6 shows the common substructure exploration system 100 has already gone through multiple iterations (e.g., 14 iterations) of selecting from the common substructure data tree.

As mentioned above, the common substructure exploration system 100 utilizes the UTC modified multi-armed bandit model for each iteration. In particular, the common substructure exploration system 100 selects one or more molecular structures from one or more data tree branches for a current iteration, generates children and mutants of the one or more selected molecular structures, and further generates measures of reward for the children, mutants, and/or the one or more selected molecular structures. In doing so, the common substructure exploration system 100 updates data tree branches for the scored molecular structures (e.g., to reflect a mean average reward of a molecular structure and its downstream mutants/children).

As alluded to above, the common substructure exploration system 100 utilizes the UCT modified multi-armed bandit model to select from the common substructure data tree. As shown in FIG. 6, the common substructure exploration system 100 utilizes the UCT modified multi-armed bandit model to compare a different data tree branches based on their corresponding measures of reward and number of visits. For example, FIG. 6 shows that the data tree branch with a measure of reward 602 of 0.7 and a number of visits 604 of 10 times. Moreover, FIG. 6 shows a data tree branch with a measure of reward 608 of 0.1 and a number of visits 610 of 4 times. Additionally, FIG. 6 shows a data tree branch with a reward 616 of unknown and a number of visits 614 as zero. In one or more embodiments, the common substructure exploration system 100 utilizes the UCT modified multi-armed bandit model to analyze the measures of reward and the number of visits and chooses from the data tree branch with the reward 616 of unknown reward.

As mentioned above in FIG. 2B, in some embodiments, the common substructure exploration system 100 selects a node for evolution, however the evolutionary children/mutants are not scored. In particular, because the selected node was not scored nor were the evolutionary children/mutants, the common substructure exploration system 100 decreases the visit count as if the particular had never been selected. In doing so, the common substructure exploration system 100 can still increase exploration by not skipping over a particular data tree branch (e.g., the bottom data tree branch shown in FIG. 6).

Although the above description describes the common substructure exploration system 100 using a UCT modified multi-armed bandit model, in one or more embodiments, the common substructure exploration system 100 obtains a prior probability distribution for a common substructure data tree. In particular, the common substructure exploration system 100 determines whether a plurality of molecular structures (e.g., obtained from a library of molecules) was selected during previous sessions. If so, the common substructure exploration system 100 relies on prior probability distributions for each “arm” (e.g., data tree branch). For example, the common substructure exploration system 100 references the prior probability distributions of reward for each data tree branch and uses that to select molecular structures from the data tree branches. In some embodiments, the common substructure exploration system 100 relies on the prior probability distribution for initially selecting from the common substructure data tree.

In one or more embodiments, the common substructure exploration system 100 tunes a parameter (e.g., a beta hyperparameter) of the multi-armed bandit model to tilt the multi-armed bandit model towards either exploration or exploitation. In particular, the common substructure exploration system 100 tunes initial rounds of selection from the common substructure data tree towards exploration and tunes later rounds of selection from the common substructure data tree towards exploitation. In some embodiments, the common substructure exploration system 100 establishes a threshold number of rounds for prioritizing exploration and a threshold number of rounds for prioritizing exploitation.

In some embodiments, the common substructure exploration system 100 utilizes an intelligent algorithmic approach to choose between exploration or exploitation. In particular, the common substructure exploration system 100 determines whether to prioritize exploration or exploitation after each iteration. For example, the common substructure exploration system 100 utilizes a machine learning model to process measure of reward(s) from previous rounds of selection and outputs a prediction of how to tune the multi-armed bandit model (e.g., 0.7 exploration and 0.3 exploitation). As mentioned above, the common substructure exploration system 100 learns from previous experience to determine how to tune the multi-armed bandit model.

In some embodiments, if the common substructure exploration system 100 selects an initial molecular structure that has a measure of reward greater than a threshold amount, then the common substructure exploration system 100 tunes the multi-armed model to prioritize exploitation (e.g., from the data tree branch where the molecular structure was selected). In some embodiments, if the common substructure exploration system 100 selects a molecular structure that has a measure of reward below a threshold amount, the common substructure exploration system 100 tunes the multi-armed model to prioritize exploration (e.g., to explore different data tree branches). As mentioned above, in some embodiments, the common substructure exploration system 100 sets the multi-armed bandit model to always select from a data tree branch of the common substructure tree that has not yet been visited.

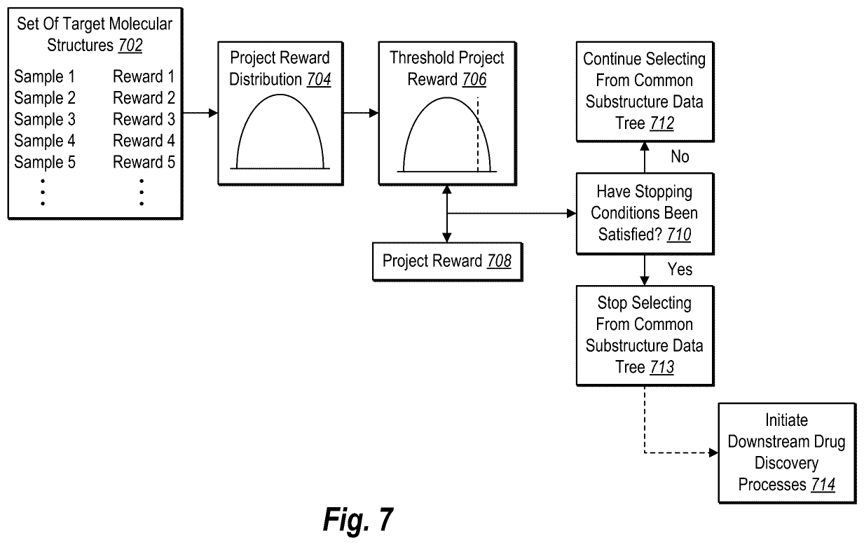

FIG. 7 illustrates an example diagram of the common substructure exploration system 100 generating a project reward distribution and determining whether stopping conditions have been satisfied in accordance with one or more embodiments. For example, FIG. 7 shows the common substructure exploration system 100 accessing a set of target molecular structures 702 and generating a project reward distribution 704 from the set of target molecular structures 702. In particular, the set of target molecular structures 702 can include any selected molecular structure from the common substructure data tree. In some instances, the common substructure exploration system 100 only uses molecular structures from the set of target molecular structures 702 that satisfy a threshold score (e.g., a threshold score for a measure of reward).

As mentioned above, in some embodiments, the common substructure exploration system 100 generates the project reward distribution 704 utilizing a Bayesian approach. For example, the common substructure exploration system 100 initially generates a set of target molecular structures (e.g., from selecting from the common substructure data tree) and leverages the data from the current project as a posterior probability to assess the current performance of selecting molecular structures from the common substructure data tree.

As shown, the common substructure exploration system 100 generates the project reward distribution 704. In particular, the common substructure exploration system 100 samples from the common substructure data tree, and based on the sampled molecular structures, the common substructure exploration system 100 generates the project reward distribution 704. In other words, the common substructure exploration system 100 creates a probability distribution of rewards for the current project (e.g., the already selected molecule structures). Thus, the common substructure exploration system 100 can refer to the project reward distribution 704 to determine how likely additional rounds of selecting from the common substructure data tree would result in increased reward.

In one or more embodiments, the common substructure exploration system 100 runs ten to twelve generations of selecting one or more molecular structures (in each round) from the common substructure data tree. Across these ten to twelve generations, the common substructure exploration system 100 can further determine to continue additional generations of selection in response to the generated project reward distribution. In other words, the common substructure exploration system 100 can be more intelligent and informed in deciding a number of rounds to select from the common substructure data tree.

In some embodiments, the common substructure exploration system 100 determines a threshold project reward 706 from the project reward distribution 704. In particular, the common substructure exploration system 100 determines the threshold project reward 706 as the maximum reward indicated by the project reward distribution 704, or the common substructure exploration system 100 determines the threshold project reward 706 as a certain percentage of the maximum reward (e.g., 90%).

In one or more embodiments, the common substructure exploration system 100 extracts a project reward 708 from the already sampled molecule structures (e.g., the set of target molecular structures 702 or an elite subset of molecular structures that satisfies a threshold score). In particular, the project reward 708 refers to a measure of reward for a particular sample. In some embodiments, the common substructure exploration system 100 selects the project reward 708 that is the highest relative to other project rewards from the already sampled molecule structures. Moreover, in some embodiments, the common substructure exploration system 100 compares the project reward 708 to the threshold project reward 706 to determine whether a stopping condition has been satisfied.

In one or more embodiments, the common substructure exploration system 100 can establish stopping conditions. In particular, stopping conditions refer to conditions of the current project (e.g., the already sampled molecular structures from the common substructure data tree) that would result in the common substructure exploration system 100 terminating additional selection rounds (e.g., iterations/generations) from the common substructure data tree. In one or more embodiments, the common substructure exploration system 100 selects a stopping condition by using the threshold project reward 706. In other words, the common substructure exploration system 100 determines that if the threshold project reward 706 has been satisfied, then the common substructure exploration system 100 stops selecting from the common substructure data tree.

As shown in FIG. 7, the common substructure exploration system 100 performs an act 710 of determining whether stopping conditions have been satisfied. Moreover, FIG. 7 shows that if the common substructure exploration system 100 determines that the stopping conditions have not been satisfied, the common substructure exploration system 100 performs an act 712 of continuing to select from the common substructure data tree.

Further, FIG. 7 shows that if the common substructure exploration system 100 determines that the stopping conditions have been satisfied, the common substructure exploration system 100 performs an act 713 of stopping selection from the common substructure data tree. Additionally, FIG. 7 shows that in some embodiments, the common substructure exploration system 100 further performs an act 714 of initiating downstream drug discovery processes. In particular, the act 714 of initiating downstream drug discovery processes includes initiating drug discovery, such as a compound exploration program to perform hit-to-lead or lead optimization of one or more molecules from the set of target molecular structures 702. For example, the common substructure exploration system 100 selects one or more molecular structures from the set of target molecular structures 702 based on measures of reward of the set of target molecular structures 702.

In some embodiments, a compound exploration program includes utilizing a target gene involved in an underlying disease and testing one or more of the set of target molecular structures 702 to identify how the compounds interact with the specific target. Additionally, compound exploration programs involve optimizing identified compounds from the set of target molecular structures 702 and analyzing results of the compounds applied to the specific target. In some instances, compound exploration programs also involve pre-clinical testing, clinical trials, candidate selection for further testing, and regulatory approval.

Moreover, the common substructure exploration system 100 initiates downstream drug discovery such as an industrial program generation (IPG) process. To illustrate, IPG includes (i) a hit selection to identify statistically strong connections based on measures of reward of the set of target molecular structures 702 to patient-informed phenotypes, (ii) phenomic confirmation (e.g., promising actives are confirmed by automated similarity and concentration-response analytics), (iii) Trekseq confirmation (e.g., compound and gene relationships are confirmed with transcriptomics in the map background based on measures of reward of the set of target molecular structures 702), and (iv) Structure-Activity Relationship (SAR) confidence (e.g., actives that behave as a series are identified, and an automated recommendation for expansion is identified). In other words, the common substructure exploration system 100 leverages measures of rewards of the set of target molecular structures 702 and designs an IPG process around one or more molecular structures.

Moreover, in some embodiments the compound exploration initiation system utilizes the measures of reward of the set of target molecular structures 702 to determine to initiate an industrialized compound generation (ICG) process. For instance, ICG applies to steps subsequent to IPG. Further, in some embodiments ICG includes rapidly searching and expanding from potential hit series in the chemical space (e.g., identified at the IPG stage) and testing the potential hits with various analytical tests (e.g., SAR screens).

In one or more embodiments, the common substructure exploration system 100 incorporates with an artificial-intelligent (AI) tech-bio system, which is described in patent application Ser. No. 19/000,315 UTILIZING LANGUAGE MACHINE LEARNING MODELS FOR AUTONOMOUS EXECUTIONS OF COMPUTERIZED TECH-BIO EXPLORATION TOOLS, which was filed on Dec. 23, 2024, and is fully incorporated by reference herein. In particular the AI tech-bio system utilizes a language machine learning model as an autonomous agent to interact with one or more computerized tech-bio exploration tools within a computerized bio-activity discovery pipeline (or other drug discovery data pipeline) to generate and/or obtain bio-activity data. In particular, the AI tech-bio system can utilize train (or configure) one or more language machine learning models as autonomous agents that learn to interact with (or utilize) one or more tools within a computerized drug discovery pipeline. For instance, the AI tech-bio system can enable an interactive application that facilitates prompt-based tech-bio queries (or requests) that cause the language machine learning model-based autonomous agents to execute one or more tasks (based on an analysis of the prompt-based tech-bio request) within the computerized bio-activity discovery pipeline to generate and/or obtain bio-activity data as a response to the prompt-based tech-bio request.

Moreover, in some instances, the AI tech-bio system can also utilize the language machine learning model-based autonomous agents to automatically execute various combinations of tasks within the computerized bio-activity discovery pipeline to identify and/or generate various bio-activity data and/or insights within a tech-bio exploration system ecosystem (e.g., compound identification, gene and compound relationship identification, protein of interest identification, compound program recommendations, perturbation image generation, perturbation heatmap generation, mechanism-of-action data).

In one or more embodiments, the AI tech-bio system communicates with the common substructure exploration system 100 to generate a text response with a novel/selected compound and/or a measure of reward from selecting a compound from a common substructure data tree. In particular, the AI tech-bio system interacts with the common substructure exploration system 100 to generate a common substructure data tree of a molecular structure population, selects one or more molecular structures, and generates one or more measures of reward. In some embodiments, the common substructure exploration system 100 generates evolved/mutated molecular structures. Further, the common substructure exploration system 100 communicates with the AI tech-bio system to provide data/information regarding the selected molecular structures.

Moreover, in some embodiments, one or more systems incorporate with the common substructure exploration system 100 and leverages the mutation/evolution components of the common substructure exploration system 100. In particular, one or more systems identify one or more input molecules and the common substructure exploration system 100 generates a common substructure data tree of the input molecules, selects molecules, and then further evolves/mutates the selected molecules. In doing so, the common substructure exploration system 100 further diversifies a starting population.

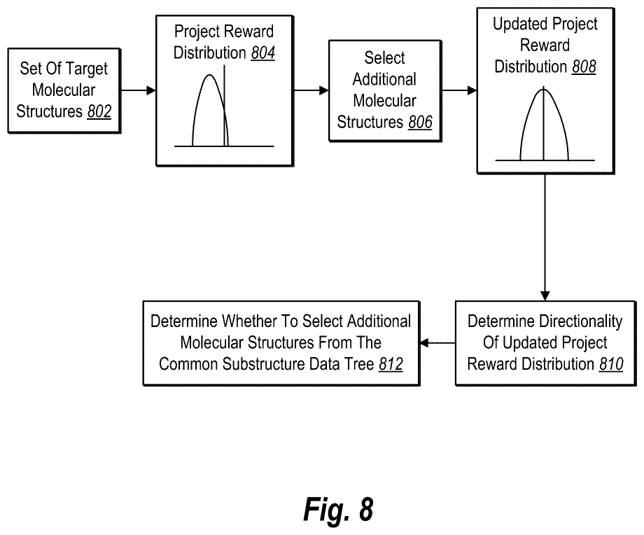

FIG. 8 illustrates an example diagram of the common substructure exploration system 100 comparing an updated project reward distribution with an initial project reward distribution in accordance with one or more embodiments. For example, FIG. 8 shows the common substructure exploration system 100 accessing a set of target molecular structures 802. Similar to FIG. 7, the common substructure exploration system 100 generates a project reward distribution 804 of the set of target molecular structures 802.

In one or more embodiments, the project reward distribution 804 is for an initial iteration of selecting from the common substructure data tree. Moreover, in some embodiments, the common substructure exploration system 100 performs an act 806 of selecting additional molecular structures from the common substructure data tree. In doing so, the common substructure exploration system 100 generates an updated project reward distribution 808. In other words, the common substructure exploration system 100 adds additional molecular structures (e.g., from selecting from the data tree and from further evolving/mutating a selected molecular structure) and creates an updated distribution of the rewards.

As shown in FIG. 8, the updated project reward distribution 808 shows that the distribution changed relative to an initial project reward distribution. In one or more embodiments, the common substructure exploration system 100 performs an act 810 of determining a directionality of the updated project reward distribution 808. In other words, the common substructure exploration system 100 compares a current project reward distribution with a prior project reward distribution to determine the direction of the distribution (e.g., is the probability distribution increasing or shrinking). In particular, the common substructure exploration system 100 performs a first iteration of selecting from the common substructure data tree, generates a first project reward distribution, performs a second iteration of selecting from the common substructure data tree, and then generates a second project reward distribution to see a trend of how the distribution is changing.

Additionally, the common substructure exploration system 100 can use the directionality to perform an act 812 of determining whether to select additional molecular structures from the common substructure data tree. In particular, if the probability distribution is increasing, the common substructure exploration system 100 can determine to continue to select additional molecular structures (e.g., perform additional iterations), whereas if the probability distribution is shrinking, the common substructure exploration system 100 can determine to stop selecting molecular structures.

In one or more embodiments, the common substructure exploration system 100 can provide for display on a graphical user interface of a computing device, a project reward distribution, a threshold project reward, stopping conditions, whether stopping conditions have been satisfied, and a directionality. In particular, the common substructure exploration system 100 can provide for display on a graphical user interface of a computing device, data related to the maximum reward/regret of performing additional iterations of selecting from a common substructure data tree. Moreover, in some embodiments, the common substructure exploration system 100 can provide one or more options for a user of a computing device to stop selection from a common substructure data tree, or for a user to override stopping conditions and to continue to select from the common substructure data tree.

In one or more embodiments, the common substructure exploration system 100 can further monitor databases/libraries of molecular structures and automatically initiate workflows in response to updates/new data. In particular, the common substructure exploration system 100 consistently monitors a database of molecules and generates probabilities of producing improved molecular structures. In response to generating a probability that satisfies a threshold increase in probability, the common substructure exploration system 100 initiates additional selections from a common substructure data tree.

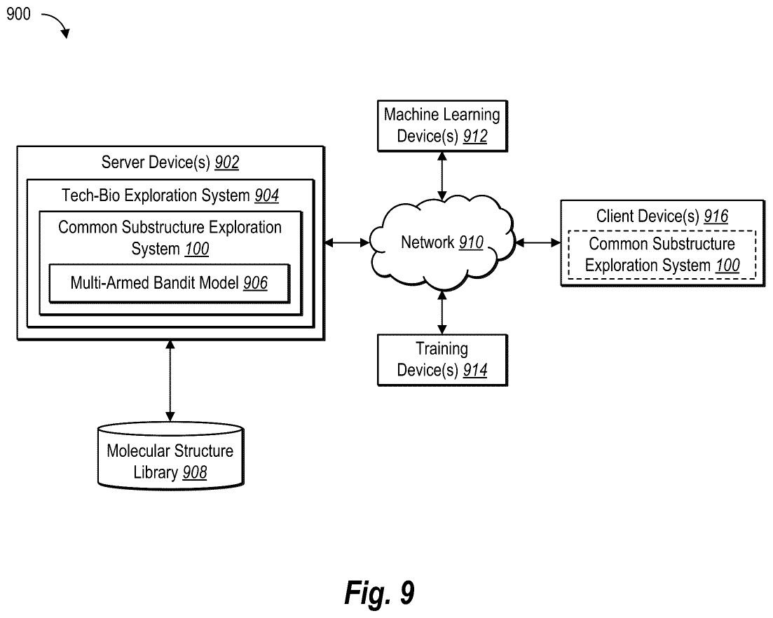

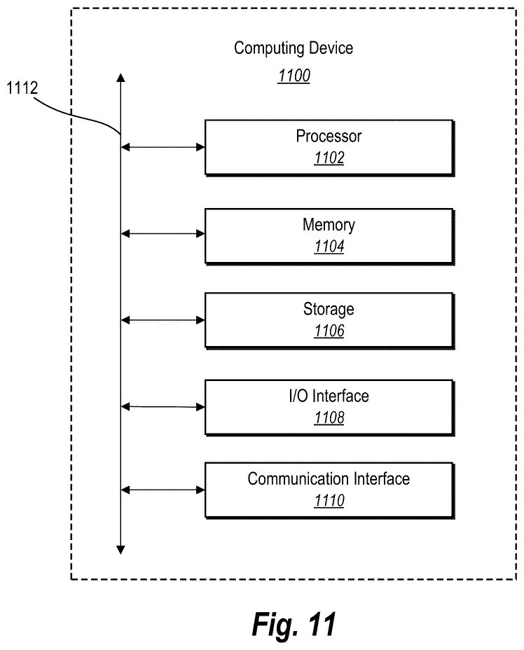

As shown in FIG. 9, an environment 900 includes server device(s) 902 (which includes a tech-bio exploration system 904 and the common substructure exploration system 100), a network 910, and client device(s) 916. As further illustrated in FIG. 9, the various computing devices within the environment 900 can communicate via the network 910. Although FIG. 9 illustrates the common substructure exploration system 100 being implemented by a particular component and/or device within the environment, the common substructure exploration system 100 can be implemented, in whole or in part, by other computing devices and/or components in the environment 900 (e.g., the additional device(s)). Additional description regarding the illustrated computing devices is provided with respect to FIG. 11 below.

As shown in FIG. 9, the server device(s) 902 (e.g., one or more local servers operated by a particular entity) can include the tech-bio exploration system 904. In some embodiments, the tech-bio exploration system 904 can determine, store, generate, and/or display tech-bio information including common substructure data trees, reward measures for selected molecular structures, evolved/mutated products of selected molecular structures, maps of biology, experiments from various sources, and/or machine learning tech-bio predictions. For instance, the tech-bio exploration system 904 can analyze selected molecular structures (e.g., and corresponding reward measures), data signals corresponding to various treatments or interventions (e.g., compounds or biologics) and the corresponding relationships in genetics, proteomics, phenomics (i.e., cellular phenotypes), and invivomics (e.g., expressions or results within a living animal). Moreover, the tech-bio exploration system 904 provides an environment for operating, executing, and managing complex drug discovery pipelines.

For instance, the tech-bio exploration system 904 can generate and access experimental results corresponding to gene sequences, protein shapes/folding, protein/compound interactions, phenotypes resulting from various interventions or perturbations (e.g., gene knockout sequences or compound treatments), and/or invivo experimentation on various treatments in living animals. By analyzing these signals (e.g., utilizing various machine learning models), the tech-bio exploration system 904 can generate or determine a variety of predictions and inter-relationships for improving treatments/interventions (e.g., generating a set of target molecular structures for initiating compound drug discovery processes).

To illustrate, the tech-bio exploration system 904 can generate a set of target molecular structures, maps of biology indicating biological inter-relationships, or similarities between these various input signals to discover potential new treatments as part of the complex compound discovery process. For example, the tech-bio exploration system 904 can utilize machine learning and/or maps of biology to identify a starting population of molecular structures, generate a common substructure data tree of the starting population, and further select one or more molecular structures from the common substructure data tree. The tech-bio exploration system 904 can then identify new programs/treatments to initiate based on selected molecular structures and their corresponding measures of reward. Similarly, the tech-bio exploration system 904 can analyze the selected molecular structures against signals from a variety of sources (e.g., protein interactions, or invivo experiments) to predict efficacious treatments based on various levels of biological data.

The tech-bio exploration system 904 can generate GUIs comprising dynamic user interface elements to convey tech-bio information and receive user input for intelligently exploring tech-bio information. Indeed, as mentioned above, the tech-bio exploration system 904 can generate GUIs displaying different maps of biology that intuitively and efficiently express complex interactions between different biological systems for identifying improved treatment solutions. Furthermore, the tech-bio exploration system 904 can also electronically communicate tech-bio information between various computing devices.

As shown in FIG. 9, the tech-bio exploration system 904 can include a system that facilitates various models or algorithms for generating a common substructure data tree. In particular, FIG. 9 shows the common substructure exploration system 100 that includes a multi-armed bandit model 906 to generate common substructure data trees. Furthermore, the tech-bio exploration system 904 can generate maps of biology (e.g., maps or visualizations illustrating similarities or relationships between genes, proteins, diseases, compounds, and/or treatments) and discovering new treatment options over one or more networks. For example, the tech-bio exploration system 904 collects, manages, and transmits data across a variety of different entities, accounts, and devices. In some cases, the tech-bio exploration system 904 is a network system that facilitates access to (and analysis of) tech-bio information within a centralized operating system. Indeed, the tech-bio exploration system 904 can link data from different network-based research institutions to generate and analyze common substructure data trees and maps of biology.

As shown in FIG. 9, the tech-bio exploration system 904 can include a system that comprises the common substructure exploration system 100 that generates, stores, manages, transmits data pertaining to molecular structures selected from a common substructure data tree which was built from a molecular structure library 908 for the chemical-reaction action space. For example, in context of the above description for the tech-bio exploration system 904, in some embodiments the tech-bio exploration system 904 further utilizes the common substructure exploration system 100 to enhance the coordination between various groups involved in the drug discovery process. For instance, the common substructure exploration system 100 works in tandem with the tech-bio exploration system 904 to generate molecular structures that indicate similarities or relationships between genes, proteins, diseases, compounds, and/or treatments and can utilize generated molecular structures to further discover new treatment options. Specifically, the tech-bio exploration system 904 utilizes the common substructure exploration system 100 to generate variations of different molecular structures based on different drug exploration objectives (e.g., generate a biochemical structure with a high binding affinity with a specific type of protein and/or generate a molecular structure and then further evolve/mutate the molecular structure).

To illustrate, the common substructure exploration system 100 utilizes the multi-armed bandit model 906 to sample molecular structures from a common substructure data tree for the chemical-reaction action space. Specifically, the common substructure exploration system 100 determines to select one or more molecular structures based on a number of visits to a data tree branch and a measure of reward of a data tree branch. As mentioned above, the common substructure exploration system 100 generates measures of reward for selecting one or more molecular structures and/or generating products (e.g., evolved/mutated) from a selected molecular structure. To further illustrate, the tech-bio exploration system 904 utilizes the common substructure exploration system 100 at the program discovery phase to identify compounds (e.g., molecular structures) that target certain genes. For instance, the common substructure exploration system 100 can test various hypotheses for how a gene is affected by a compound and utilizes the multi-armed bandit model 906 to explore a large state space to efficiently learn active learning targets. Moreover, in some embodiments, the tech-bio exploration system 904 utilizes the common substructure exploration system 100 at the hit-to-lead phase (e.g., where a set of feasible compounds have already been identified) and performs additional iterations of the feasible compounds to refine the set of feasible compounds (e.g., narrow down the list by exploring the state space and prioritizing exploration or prioritizing greedier actions, e.g., higher reward actions).

As also illustrated in FIG. 9, the environment includes the client device(s) 916. As mentioned above, the client device(s) 916 can be involved in the process of drug discovery. Thus, for example, the client device(s) 916 can coordinate/manage generating a particular molecular structure along with additional mode variations of the molecular structure for further downstream testing. For instance, the client device(s) 916 can coordinate/manage testing generated molecular structures under various conditions to further determine whether to initiate one or more programs (industrial program generation or industrial compound generation) for one or more of the generated molecular structures.

To illustrate, the client device(s) 916 can include computing devices that implement or manage a compound program generation stage of a compound discovery process. Similarly, the client device(s) 916 can include computing devices that implement or manage a compound lead generation stage and the client device(s) 916 can include computing devices that implement or manage a compound/dose selection stage. For example, the common substructure exploration system 100 can receive one or more requests to generate one or more molecular structures according to an input state and an objective for that input state.

In some embodiments, the environment also includes additional device(s). For example, the common substructure exploration system 100 can utilize the additional device(s) to further operate and manage downstream operations after generating one or more molecular structures. For instance, the additional device(s) include experimental device(s) and analytical device(s). Further, in some instances, the additional device(s) also include the computing devices discussed below in FIG. 11.

Furthermore, in one or more implementations, the client device(s) 916 include a client application. The client application can include instructions that (upon execution) cause the client device(s) 916 to perform various actions. For example, a user of a user account can interact with the client application on the client device(s) 916 to execute the generation of a set of target molecular structures from a common substructure data tree (e.g., or other non-biochemical structures) by exploring a state space and executing experiments or other multi-faceted processes based on generated (e.g., selected) molecular structures. For instance, in some embodiments the common substructure exploration system 100 receives a request to generate a set of target molecular structure from an input population and an objective for the input population. In response, the common substructure exploration system 100 can further generate one or more target molecular structures according to the objective and returns the molecular structure to the client device(s) 914 along with corresponding measures of reward. In some instances, the transmittal of the target molecular structure to the client device(s) 914 causes the client device(s) 914 to further present options for executing an action (e.g., performing downstream experiments, tests, or evaluations of the generated molecular structure).

In one or more embodiments, the environment 900 can also include dedicated training device(s) 914. For example, the dedicated training device(s) 914 can include computing devices or virtual machines dedicated to training or implementing a multi-armed bandit model 906 and/or machine learning models used to generate measures of reward. For example, the dedicated training device(s) 914 can provide datasets, parameters, objectives, and other learning constraints to train the machine learning models to generate outputs specific to a task. Thus, the common substructure exploration system 100 interacts with the dedicated training device(s) 914 to learn certain state spaces and to accurately generate corresponding measures of reward.

As shown in FIG. 9, the environment 900 includes machine learning device(s) 912. In particular, the machine learning device(s) 912 are attuned for specific objectives (e.g., reward configurations), and the common substructure exploration system 100 accesses the training device(s) 914 to train/fine-tune the machine learning device(s) 912. In some embodiments, the common substructure exploration system 100 utilizes the machine learning device(s) 912 to generate a starting population of molecular structures.

The environment can also include experimental device(s). For example, the tech-bio exploration system 904 can interact with the experimental device(s) that include intelligent robotic devices and camera devices for generating and capturing digital images of cellular phenotypes resulting from different perturbations (e.g., genetic knockouts or compound treatments of stem cells). Similarly, the experimental device(s) can include camera devices and/or other sensors (e.g., heat or motion sensors) capturing real-time information from animals as part of invivo experimentation. The tech-bio exploration system 904 can also interact with a variety of other experimental device(s) such as devices for determining, generating, or extracting gene sequences or protein information. For example, the experimental device(s) may include computing devices linked to biosensorselectrophysiological platforms, x-ray crystallography machines, liquid chromatography mass spectrometry systems, nuclear magnetic resonance spectrometers, mass spectrometers. In some implementations, the common substructure exploration system 100 generates tractability scores and further determines to employ or utilize one or more experimental devices (e.g., to initiate one or more experiments based on the tractability scores).

As further shown in FIG. 9, the environment includes the network 910. As mentioned above, the network 910 can enable communication between components of the environment. In one or more embodiments, the network 910 may include a suitable network and may communicate using a various number of communication platforms and technologies suitable for transmitting data and/or communication signals, examples of which are described with reference to FIG. 11. Furthermore, although FIG. 9 illustrates computing devices communicating via the network 910, the various components of the environment can communicate and/or interact via other methods (e.g., communicate directly).

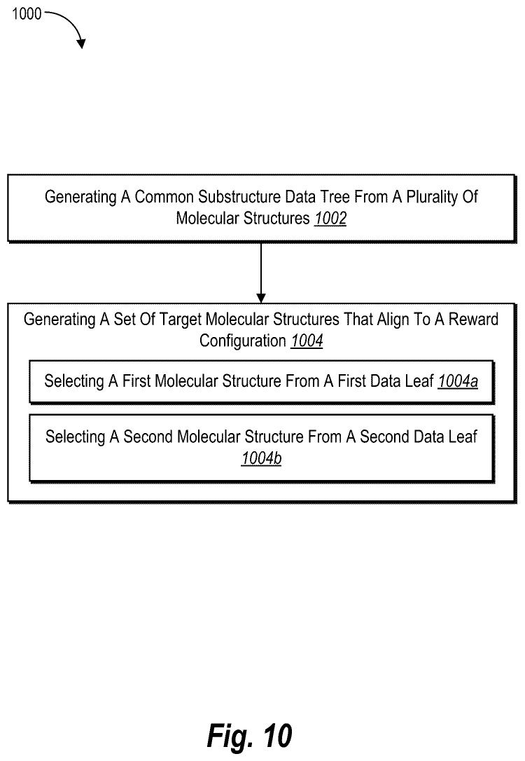

FIGS. 1-9, the corresponding text, and the examples provide a number of different systems, methods, and non-transitory computer readable media for utilizing common substructure exploration system 100 to generate a set of target molecular structures. In addition to the foregoing, embodiments can also be described in terms of flowcharts comprising acts for accomplishing a particular result. For example, FIG. 10 illustrates a flowchart of an example sequence of acts in accordance with one or more embodiments.

While FIG. 10 illustrates acts according to some embodiments, alternative embodiments may omit, add to, reorder, and/or modify any of the acts shown in FIG. 10. The acts of FIG. 10 can be performed as part of a method (e.g., a computer-implemented method). Alternatively, a non-transitory computer readable medium can comprise instructions, that when executed by one or more processors (e.g., at least one processor), cause a computing device to perform the acts of FIG. 10. In still further embodiments, a system can perform the acts of FIG. 10. Additionally, the acts described herein may be repeated or performed in parallel with one another or in parallel with different instances of the same or other similar acts.

FIG. 10 illustrates an example series of acts 1000 for generating a set of target molecular structures that align to a reward configuration in accordance with one or more embodiments. The series of acts 1000 can include an act 1002 of generating a common substructure data tree from a plurality of molecular structures, an act 1004 of generating a set of target molecular structures that align to a reward configuration, a sub-act 1004a of selecting a first molecular structure from a first data leaf, and a sub-act 1004b of selecting a second molecular structure from a second data leaf.

Specifically, the series of acts 1000 can include acts of generating, from a plurality of molecular structures, a common substructure data tree by generating a plurality of data tree branches having a plurality of data leaves corresponding to the plurality of molecular structures, wherein the plurality of data tree branches indicate common molecular substructures of data leaves within the plurality of data tree branches; generating a set of target molecular structures that align to a reward configuration by: selecting, utilizing a multi-armed bandit model, a first molecular structure from a first data leaf within a first data tree branch of the common substructure data tree; and selecting, utilizing the multi-armed bandit model, a second molecular structure from a second data leaf of the common substructure data tree based on a number of leaves previously selected from the first data tree branch.

In one or more embodiments, the series of acts 1000 includes a reward machine learning model, further comprising generating, utilizing the reward machine learning model, a measure of reward for the first molecular structure. In one or more implementations, the series of acts 1000 includes selecting the reward configuration from a plurality of reward configurations, wherein the plurality of reward configurations comprise a plurality of objectives to improve in selecting target molecular structures.

In one or more embodiments, the series of acts 1000 includes selecting the second molecular structure based on a measure of reward for the first molecular structure corresponding to the reward configuration. In addition, in one or more implementations, the series of acts 1000 includes determining that the first data tree branch has been previously selected and that the second molecular structure from the second data leaf is part of the first data tree branch; and based on comparing a measure of reward for the first molecular structure and additional measures of reward of additional data tree branches of the common substructure data tree, selecting the second molecular structure.

Further, in some implementations, the series of acts 1000 includes determining that the second molecular structure from the second data leaf is part of a second data tree branch that has not been previously selected; and based on the second data tree branch not being previously selected and the first data tree branch being previously selected, selecting the second molecular structure from the second data leaf of the second data tree branch.

In one or more implementations, the series of acts 1000 includes generating a reward measure for the second molecular structure corresponding to the reward configuration; and based on the second data tree branch having been previously selected and the first data tree branch having been previously selected, selecting a third molecular structure from a third leaf of a third data tree branch. Moreover, in one or more implementations, the series of acts 1000 includes generating, utilizing a sexual selection algorithm, a plurality of children molecular structures from the set of target molecular structures; generating, utilizing a mutation algorithm, a plurality of mutant molecular structures from the set of target molecular structures; and selecting one or more molecular structures from the plurality of children molecular structures, the plurality of mutant molecular structures, or the set of target molecular structures to utilize for downstream drug discovery purposes.

In addition, in some implementations, the series of acts 1000 includes generating based on sampled molecular structures from the common substructure data tree, a project reward distribution for the reward configuration; determining, from the project reward distribution, a threshold project reward; selecting a stopping condition utilizing the threshold project reward; and determining whether the stopping condition has been satisfied by comparing a project reward for the sampled molecular structures to the threshold project reward. In one or more implementations, the series of acts 1000 includes determining a directionality of a project reward distribution upon iteratively selecting from the common substructure data tree; and utilizing the directionality to determine whether to select additional molecular structures from the common substructure data tree.

Moreover, in one or more implementations, the series of acts 1000 includes generating based on sampled molecular structures from the common substructure data tree, a project reward distribution for the reward configuration; determining, from the project reward distribution, a threshold project reward; and selecting a stopping condition utilizing the threshold project reward.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 11 illustrates a block diagram of an example computing device 1100 that may be configured to perform one or more of the processes described above. One will appreciate that one or more computing devices, such as the computing device 1100 may represent the computing devices described above. In one or more embodiments, the computing device 1100 may be a mobile device (e.g., a mobile telephone, a smartphone, a PDA, a tablet, a laptop, a camera, a tracker, a watch, a wearable device, etc.). In some embodiments, the computing device 1100 may be a non-mobile device (e.g., a desktop computer or another type of client device). Further, the computing device 1100 may be a server device that includes cloud-based processing and storage capabilities.

As shown in FIG. 11, the computing device 1100 can include one or more processor(s) 1102, memory 1104, a storage device 1106, input/output interfaces 1108 (or “I/O interfaces 1108”), and a communication interface 1110, which may be communicatively coupled by way of a communication infrastructure (e.g., bus 1112). While the computing device 1100 is shown in FIG. 11, the components illustrated in FIG. 11 are not intended to be limiting. Additional or alternative components may be used in other embodiments. Furthermore, in certain embodiments, the computing device 1100 includes fewer components than those shown in FIG. 11. Components of the computing device 1100 shown in FIG. 11 will now be described in additional detail.

In particular embodiments, the processor(s) 1102 includes hardware for executing instructions, such as those making up a computer program. As an example, and not by way of limitation, to execute instructions, the processor(s) 1102 may retrieve (or fetch) the instructions from an internal register, an internal cache, memory 1104, or a storage device 1106 and decode and execute them.

The computing device 1100 includes memory 1104, which is coupled to the processor(s) 1102. The memory 1104 may be used for storing data, metadata, and programs for execution by the processor(s). The memory 1104 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1104 may be internal or distributed memory.

The computing device 1100 includes a storage device 1106 includes storage for storing data or instructions. As an example, and not by way of limitation, the storage device 1106 can include a non-transitory storage medium described above. The storage device 1106 may include a hard disk drive (HDD), flash memory, a Universal Serial Bus (USB) drive or a combination these or other storage devices.

As shown, the computing device 1100 includes one or more I/O interfaces 1108, which are provided to allow a user to provide input to (such as user strokes), receive output from, and otherwise transfer data to and from the computing device 1100. These I/O interfaces 1108 may include a mouse, keypad or a keyboard, a touch screen, camera, optical scanner, network interface, modem, other known I/O devices or a combination of such I/O interfaces 1108. The touch screen may be activated with a stylus or a finger.

The I/O interfaces 1108 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1108 are configured to provide graphical data to a display for presentation to a user. The graphical data may be representative of one or more graphical user interfaces and/or any other graphical content as may serve a particular implementation.

The computing device 1100 can further include a communication interface 1110. The communication interface 1110 can include hardware, software, or both. The communication interface 1110 provides one or more interfaces for communication (such as, for example, packet-based communication) between the computing device and one or more other computing devices or one or more networks. As an example, and not by way of limitation, communication interface 1110 may include a network interface controller (NIC) or network adapter for communicating with an Ethernet or other wire-based network or a wireless NIC (WNIC) or wireless adapter for communicating with a wireless network, such as a WI-FI. The computing device 1100 can further include a bus 1112. The bus 1112 can include hardware, software, or both that connects components of computing device 1100 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 1100 can include a client device that includes a requester application or a web browser, such as MICROSOFT INTERNET EXPLORER, GOOGLE CHROME, or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 904 may include a variety of servers, sub-systems, programs, modules, logs, and data stores. In particular embodiments, the tech-bio exploration system 904 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 904 may also include suitable components such as network interfaces, security mechanisms, load balancers, failover servers, management-and-network-operations consoles, other suitable components, or any suitable combination thereof. In particular embodiments, the tech-bio exploration system 904 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 904 and one or more client devices. An action logger may be used to receive communications from a web server about a user's actions on or off the tech-bio exploration system 904. In conjunction with the action log, a third-party-content-object log may be maintained of user exposures to third-party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 904. A privacy setting of a user determines how particular information associated with a user can be shared. The authorization server may allow users to opt in to or opt out of having their actions logged by the tech-bio exploration system 904 or shared with other systems, such as, for example, by setting appropriate privacy settings. Third-party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
generating, from a plurality of molecular structures, a common substructure data tree by generating a plurality of data tree branches having a plurality of data leaves corresponding to the plurality of molecular structures, wherein the plurality of data tree branches indicate common molecular substructures of data leaves within the plurality of data tree branches; and
generating a set of target molecular structures that align to a reward configuration by:
selecting, utilizing a multi-armed bandit model, a first molecular structure from a first data leaf within a first data tree branch of the common substructure data tree; and
selecting, utilizing the multi-armed bandit model, a second molecular structure from a second data leaf of the common substructure data tree based on a number of leaves previously selected from the first data tree branch.

2. The computer-implemented method of claim 1, wherein the reward configuration comprises a reward machine learning model, further comprising generating, utilizing the reward machine learning model, a measure of reward for the first molecular structure.

3. The computer-implemented method of claim 1, further comprising selecting the reward configuration from a plurality of reward configurations, wherein the plurality of reward configurations comprises a plurality of objectives to improve in selecting target molecular structures.

4. The computer-implemented method of claim 1, wherein selecting the second molecular structure from the second data leaf comprises selecting the second molecular structure based on a measure of reward for the first molecular structure corresponding to the reward configuration.

5. The computer-implemented method of claim 1, wherein selecting the second molecular structure from the second data leaf comprises:
determining that the second molecular structure from the second data leaf is part of a second data tree branch that has not been previously selected; and
based on the second data tree branch not being previously selected and the first data tree branch being previously selected, selecting the second molecular structure from the second data leaf of the second data tree branch.

6. The computer-implemented method of claim 5, further comprising:
generating a reward measure for the second molecular structure corresponding to the reward configuration; and
based on the second data tree branch having been previously selected and the first data tree branch having been previously selected, selecting a third molecular structure from a third leaf of a third data tree branch.

7. The computer-implemented method of claim 1, further comprising:
generating, utilizing a sexual selection algorithm, a plurality of children molecular structures from the set of target molecular structures;
generating, utilizing a mutation algorithm, a plurality of mutant molecular structures from the set of target molecular structures; and
selecting one or more molecular structures from the plurality of children molecular structures, the plurality of mutant molecular structures, or the set of target molecular structures to utilize for downstream drug discovery purposes.

8. The computer-implemented method of claim 1, further comprising:
generating based on sampled molecular structures from the common substructure data tree, a project reward distribution for the reward configuration;
determining, from the project reward distribution, a threshold project reward;
selecting a stopping condition utilizing the threshold project reward; and
determining whether the stopping condition has been satisfied by comparing a project reward for the sampled molecular structures to the threshold project reward.

9. The computer-implemented method of claim 1, further comprising:
determining a directionality of a project reward distribution upon iteratively selecting from the common substructure data tree; and
utilizing the directionality to determine whether to select additional molecular structures from the common substructure data tree.

10. A system comprising:
at least one processor; and
at least one non-transitory computer-readable storage medium storing instructions that, when executed by the at least one processor, cause the system to:
generate, from a plurality of molecular structures, a common substructure data tree by generating a plurality of data tree branches having a plurality of data leaves corresponding to the plurality of molecular structures, wherein the plurality of data tree branches indicate common molecular substructures of data leaves within the plurality of data tree branches; and
generate a set of target molecular structures that align to a reward configuration by:
selecting, utilizing a multi-armed bandit model, a first molecular structure from a first data leaf within a first data tree branch of the common substructure data tree; and
selecting, utilizing the multi-armed bandit model, a second molecular structure from a second data leaf of the common substructure data tree based on a number of leaves previously selected from the first data tree branch.

11. The system of claim 10, further comprising instructions that, when executed by the at least one processor, cause the system to generate, utilizing a reward machine learning model, a measure of reward for the first molecular structure.

12. The system of claim 10, further comprising instructions that, when executed by the at least one processor, cause the system to select the reward configuration from a plurality of reward configurations, wherein the plurality of reward configurations comprise a plurality of objectives to improve in selecting target molecular structures.

13. The system of claim 10, further comprising instructions that, when executed by the at least one processor, cause the system to select the second molecular structure from the second data leaf by:
determining that the first data tree branch has been previously selected and that the second molecular structure from the second data leaf is part of the first data tree branch; and
based on comparing a measure of reward for the first molecular structure and additional measures of reward of additional data tree branches of the common substructure data tree, selecting the second molecular structure.

14. The system of claim 10, further comprising instructions that, when executed by the at least one processor, cause the system to select the second molecular structure from the second data leaf by:
determining that the second molecular structure from the second data leaf is part of a second data tree branch that has not been previously selected; and
based on the second data tree branch not being previously selected and the first data tree branch being previously selected, selecting the second molecular structure from the second data leaf of the second data tree branch.

15. The system of claim 10, further comprising instructions that, when executed by the at least one processor, cause the system to:
generate based on sampled molecular structures from the common substructure data tree, a project reward distribution for the reward configuration;
determine, from the project reward distribution, a threshold project reward; and
select a stopping condition utilizing the threshold project reward.

16. A non-transitory computer-readable medium storing instructions that, when executed by at least one processor, cause a computing device to:
generate, from a plurality of molecular structures, a common substructure data tree by generating a plurality of data tree branches having a plurality of data leaves corresponding to the plurality of molecular structures, wherein the plurality of data tree branches indicate common molecular substructures of data leaves within the plurality of data tree branches; and
generate a set of target molecular structures that align to a reward configuration by:
selecting, utilizing a multi-armed bandit model, a first molecular structure from a first data leaf within a first data tree branch of the common substructure data tree; and
selecting, utilizing the multi-armed bandit model, a second molecular structure from a second data leaf of the common substructure data tree based on a number of leaves previously selected from the first data tree branch.

17. The non-transitory computer-readable medium of claim 16, further comprising instructions that, when executed by the at least one processor, cause the computing device to select the second molecular structure from the second data leaf by:
determining that the first data tree branch has been previously selected and that the second molecular structure from the second data leaf is part of the first data tree branch; and
based on comparing a measure of reward for the first molecular structure and additional measures of reward of additional data tree branches of the common substructure data tree, selecting the second molecular structure.

18. The non-transitory computer-readable medium of claim 16, further comprising instructions that, when executed by the at least one processor, cause the computing device to select the second molecular structure from the second data leaf by:
determining that the second molecular structure from the second data leaf is part of a second data tree branch that has not been previously selected; and
based on the second data tree branch not being previously selected and the first data tree branch being previously selected, selecting the second molecular structure from the second data leaf of the second data tree branch.

19. The non-transitory computer-readable medium of claim 16, further comprising instructions that, when executed by the at least one processor, cause the computing device to:
generate based on sampled molecular structures from the common substructure data tree, a project reward distribution for the reward configuration;
determine, from the project reward distribution, a threshold project reward; and
select a stopping condition utilizing the threshold project reward.

20. The non-transitory computer-readable medium of claim 16, further comprising instructions that, when executed by the at least one processor, cause the computing device to:
determine a directionality of a project reward distribution upon iteratively selecting from the common substructure data tree; and
utilize the directionality to determine whether to select additional molecular structures from the common substructure data tree.
